(19) United States
(12) Patent Application Publication
(10) Pub. No.: US 2021/0062150 A1
(43) Pub. Date: Mar. 4, 2021

(54) METHODS OF PREPARING T CELLS FOR T CELL THERAPY

(71) Applicant: Allogene Therapeutics, Inc., South San Francisco, CA (US)

(72) Inventors: Yajin NI, San Diego, CA (US); Chupei ZHANG, San Francisco, CA (US); Mark W. LEONARD, Burlingame, CA (US); Thomas Charles PERTEL, San Mateo, CA (US)

(21) Appl. No.: 17/010,707

(22) Filed: Sep. 2, 2020

Related U.S. Application Data
(60) Provisional application No. 62/895,381, filed on Sep. 3, 2019.

(51) Int. Cl. C12N 5/0783

(52) U.S. Cl. CPC C12N 5/0636; C12N 2501/998; C12N 2506/11

(57) ABSTRACT

Provided herein are methods for preparing T cells for T cell therapy comprising contacting a cell population at a predetermined cell density, with a concentration of an anti-CD3/CD28 nanomatrix and culturing the cells thereby producing a T cell population comprising an increased percentage of at least one T cell subtype. In some embodiments, the method increases the percentage of stem memory T cells.

### CROSS REFERENCE TO RELATED APPLICATIONS

The present application claims the benefit of priority to U.S. Provisional Application No. 62/895,381, filed Sep. 3, 2019, the contents of which are hereby incorporated by reference in their entireties.

### FIELD OF THE INVENTION

The instant disclosure relates to methods of preparing one or more T cells for a T cell therapy. In particular, the instant disclosure relates to a method of increasing the percentage of at least one T cell subtype in a cell population by contacting a cell population having a predetermined cell density with a concentration of an anti-CD3/CD28 nanomatrix selected to increase the percentage of the T cell subtype.

### BACKGROUND

Human cancers are by their nature comprised of normal cells that have undergone a genetic or epigenetic conversion to become abnormal cancer cells. In doing so, cancer cells begin to express proteins and other antigens that are distinct from those expressed by normal cells. These aberrant tumor antigens can be used by the body's innate immune system to specifically target and kill cancer cells. However, cancer cells employ various mechanisms to prevent immune cells, such as T and B lymphocytes, from successfully targeting cancer cells.

Human T cell therapies rely on ex vivo-enriched or modified human T cells to target and kill cancer cells in a subject, e.g., a patient. Various technologies have been developed to enrich the concentration of naturally occurring T cells capable of targeting a tumor antigen or genetically modifying T cells to specifically target a known cancer antigen. These therapies have proven to have promising effects on tumor size and patient survival.

Transplantation of a mixed population of T cells is among the factors that may hinder T cell therapies from reaching their full potential. In conventional T cell therapies, donor T cells are collected, optionally modified to target a specific antigen (e.g., a tumor cell) or selected for anti-tumor characteristics (e.g., tumor infiltrating lymphocytes), expanded in vitro, and administered to a subject in need thereof. Typically, the resulting T cells comprise a mixed population of largely mature cells, many of which are terminally differentiated. As a result, the expected in vivo persistence of these cells can be limited, and positive effects initially observed can be undone over time as tumors rebound in the absence of transplanted T cells. Thus, there remains a need to increase the in vivo persistence of T cells for use in a T cell therapy.

### SUMMARY OF THE INVENTION

The present disclosure provides a method for increasing a percentage of stem memory T cells in a cell population comprising 1) contacting a volume of an anti-CD3/CD28 nanomatrix with a volume of a starting cell population at a volumetric ratio wherein the starting cell population is at a cell density of at least about 0.2×10⁶ cells/ml to about 5.8×10⁶ cells/ml and the volumetric ratio is 1 volume of anti-CD3/CD28 nanomatrix to 17.4 volumes or less of the starting cell population, and 2) culturing the cell population in a culture medium, wherein the resulting T cell population comprises an increased percentage of stem memory T cells relative to a second T cell population wherein the second starting cell population of the same cell density is contacted with the anti-CD3/CD28 nanomatrix at a ratio of one volume of anti-CD3/CD28 nanomatrix to 17.5 or more volumes of the second starting cell population.

The present disclosure provides a method for increasing a percentage of stem memory T cells in a cell population comprising 1) contacting a volume of an anti-CD3/CD28 nanomatrix with a volume of a starting cell population at a volumetric ratio wherein the starting cell population is at a cell density of at least about 0.2×10⁶ cells/ml to about 5.8×10⁶ cells/ml and the volumetric ratio is 1 volume of anti-CD3/CD28 nanomatrix to 17.4 volumes or less of the starting cell population, and 2) culturing the cell population in a culture medium, wherein the resulting T cell population comprises an increased percentage of stem memory T cells relative to a second T cell population wherein the second starting cell population of the same cell density is contacted with the anti-CD3/CD28 nanomatrix at a ratio of one volume of anti-CD3/CD28 nanomatrix to 17.5 or more volumes of the second starting cell population further comprising transducing and/or transfecting the starting cell populations before, during or after culturing the starting cell populations with the anti-CD3/CD28 nanomatrix.

The present disclosure further provides a method for increasing a percentage of stem memory T cells in a PBMC population comprising, 1) contacting a volume of an anti-CD3/CD28 nanomatrix with a volume of a starting PBMC population at a volumetric ratio wherein the starting PBMC population is at a cell density of at least about 0.50×10⁶ cells/ml to about 2.00×10⁶ cells/ml and the volumetric ratio is 1 volume of anti-CD3/CD28 nanomatrix to 5 or 10 volumes of the starting cell population, and 2) culturing the PBMC population in a culture medium for 14 to 18 days, wherein the resulting PBMC population comprises an increased percentage of stem memory T cells relative to a second PBMC population wherein the second starting PBMC population of the same cell density is contacted with the anti-CD3/CD28 nanomatrix at a ratio of one volume of anti-CD3/CD28 nanomatrix to 20 or 40 volumes of the second starting PBMC population and cultured for the same number of days.

The present disclosure further provides a method for increasing a percentage of stem memory T cells in a purified T cell population comprising, 1) contacting a volume of an anti-CD3/CD28 nanomatrix with a volume of a starting purified T cell population at a volumetric ratio wherein he starting T cell population is at a cell density of at least about 0.80×10⁶ cells/ml to about 1.60×10⁶ cells/ml and the volumetric ratio is 1 volume of anti-CD3/CD28 nanomatrix to 10 or 15 volumes of the starting cell population, and 2) culturing the purified T cell population in a culture medium for 11 to 18 days, wherein the resulting purified T cell population comprises an increased percentage of stem memory T cells relative to a second purified T cell population wherein the second starting purified T cell population of the same cell density is contacted with the anti-CD3/CD28 nanomatrix at a ratio of one volume of anti-CD3/CD28 nanomatrix to 20, 25 or 50 volumes of the second starting T cell population and cultured for the same number of days.

The disclosure further provides method of treatment comprising the administering a therapeutically effective amount of the resulting Tscm-enriched T cell population to a subject in need thereof.

### BRIEF DESCRIPTION OF THE DRAWINGS

FIG. 1 is a bar graph showing overall actual cell yield from day 1 through day 18 for cells activated with the indicated Transact™ to cell culture volumetric ratios and the relevant controls.

FIG. 2 is a bar graph showing actual cell yield from day 1 through day 8 prior to culturing cells in G-Rex® for cells activated with the indicated Transact™ to cell culture volumetric ratios and the relevant controls.

FIG. 3 is a bar graph showing actual cell yield in G-Rex® from day 8 to day 18 for cells activated with the indicated Transact™ to cell culture volumetric ratios and the relevant controls.

FIG. 4 is a graph plotting cell expansion folds against days in process showing total cell expansion folds for cells activated with the indicated Transact™ to cell culture volumetric ratios and the relevant controls.

FIG. 5 is a bar graph and table depicting expansion folds for Day 0 to Day 1, Day 1 to Day 4, Day 4 to Day 6, Day 6 to Day 8, and Day 8 to Day 18, for cells activated with the indicated Transact™ to cell culture volumetric ratios and the relevant controls.

FIG. 6 is a bar graph depicting expansion folds for Day 0 to Day 1, Day 1 to Day 4, Day 4 to Day 6, Day 6 to Day 8, prior to expansion in G-Rex®, for cells activated with the indicated Transact™ to cell culture volumetric ratios and the relevant controls.

FIG. 7 is a bar graph showing the percentage of PBMC cell subsets on day 18 of the process for cells activated with the indicated Transact™ to cell culture volumetric ratios and the relevant controls.

FIG. 8 is a bar graph showing the proportion of CD5⁺ T cell memory subsets (having CD45RO and CD62L markers) on day 18 of the process for cells activated with the indicated Transact™ to cell culture volumetric ratios and the relevant controls.

FIG. 9 is a bar graph showing the percentage of CD4⁺ and CD8⁺ cell subsets on day 18 of the process for cells activated with the indicated Transact™ to cell culture volumetric ratios and the relevant controls.

FIG. 10A is a bar graph showing the percentage of subsets of the total CD5⁺ T cell subset using CD45RA and CD62L markers on day 18 of the process for cells activated with the indicated Transact™ to cell culture volumetric ratios and the relevant controls.

FIGS. 10B to 10H are contour plots with CD62L on the vertical axis and CD45RA on the horizontal axis showing percentages of CD5⁺ T cell subsets for cells activated with the indicated Transact™ to cell culture volumetric ratios and the relevant controls.

FIG. 11A is a bar graph showing the percentage of subsets of the total CD5⁺ T cell subset using CD45RO and CD62L markers on day 18 of the process for cells activated with the indicated Transact™ to cell culture volumetric ratios and the relevant controls.

FIGS. 11B to 11H are contour plots with CD62L on the vertical axis and CD45RA on the horizontal axis and showing percentages of CD5⁺ T cell subsets for cells activated with the indicated Transact™ to cell culture volumetric ratios and the relevant controls.

FIG. 12 is a bar graph showing percentage of CAR⁺ cells and TCR α/β-cells on day 18 for cells activated with the indicated Transact™ to cell culture volumetric ratios and the relevant controls.

FIG. 13A is a graph plotting the cell viability percentage against the day in process for cells activated with the indicated Transact™ to cell culture volumetric ratios and the relevant controls.

FIG. 13B is a graph plotting cell diameter against the day in process for cells activated with the indicated Transact™ to cell culture volumetric ratios and the relevant controls.

FIGS. 14A and 14B are graphs plotting optimal T cell expansion achieved with 1:10 and 1:15 TransAct™ dilutions. FMC63-41BB-CD3ζ anti-CD19 T cells, and UT control T cells, from two different donors were stimulated with either T Cell Activation/Expansion Kit beads (Miltenyi Biotec) or with T Cell TransAct™ polymeric nanomatrix (Miltenyi Biotec) at the indicated bead:cell or volume:volume ratios, respectively. Total T cell counts were taken at Day 9 and Day 14 post-activation (Day 0) and the fold expansion was calculated. 561=donor number; 658=donor number; UT=non-transduced T cells; M=T Cell Activation/Expansion Kit; T=TransAct™; FMC=FMC63-41BB-CD3ζ anti-CD19 CAR T cells; D9=Day 9; D14=Day 14.

FIGS. 15A and 15B are bar graphs showing TransAct™ dilution does not alter percentage of CAR⁺ T cells at end of the manufacturing process. FMC63-41BB-CD3ζ anti-CD19 T cells from two different donors were stimulated with either T Cell Activation/Expansion Kit beads (Miltenyi Biotec) or with T Cell TransAct™ polymeric nanomatrix (Miltenyi Biotec) at the indicated bead:cell or volume:volume ratios, respectively. The percentage of CAR⁺ T cells at Day 14 was determined by flow cytometry. 561=donor number; 658=donor number; Miltenyi=T Cell Activation/Expansion Kit; T=TransAct™; D14=Day 14; CAR=chimeric antigen receptor.

FIGS. 16A and 16B are bar graphs showing low concentrations of TransAct™ increase the percentage of CD4⁺ CAR T cells in the final product. FMC63-41BB-CD3ζ anti-CD19 T cells, and UT control T cells, from two different donors were stimulated with either T Cell Activation/Expansion Kit beads (Miltenyi Biotec) or with T Cell TransAct™ polymeric nanomatrix (Miltenyi Biotec) at the indicated bead:cell or volume:volume ratios, respectively. The percentage of CD4⁺ and CD8⁺ T cells at Day 14 was determined by flow cytometry. 561=donor number; 658=donor number; UT=non-transduced T cells; M=T Cell Activation/Expansion Kit; T=TransAct™; FMC=FMC63-41BB-CD3ζ anti-CD19 CAR T cells; D14=Day 14.

FIGS. 17A to 17D are bar graphs showing 1:10 and 1:15 TransAct™ dilutions result in lower percentage of CD25⁺ CAR T cells in the final product. FMC63-41BB-CD3ζ anti-CD19 T cells, and UT control T cells, from two different donors were stimulated with either T Cell Activation/Expansion Kit beads (Miltenyi Biotec) or with T Cell TransAct™ polymeric nanomatrix (Miltenyi Biotec) at the indicated bead:cell or volume:volume ratios, respectively. The percentage of CD25⁺ and 4-1BB⁺ T cells at Day 14 was determined by flow cytometry. 561=donor number; 658=donor number; UT=non-transduced T cells; M=T Cell Activation/Expansion Kit; T=TransAct™; FMC=FMC63-41BB-CD3ζ anti-CD19 CAR T cells; D14=Day 14.

FIGS. 18A to 18D are bar graphs showing 1:10 and 1:15 TransAct™ dilutions preserve T_{SCM} cells in both CD4⁺ and CD8⁺ CAR T cells, and T_{CM} cells in CD8⁺ CAR T cells. FMC63-41BB-CD3ζ anti-CD19 T cells, and UT control T cells, from two different donors were stimulated with either T Cell Activation/Expansion Kit beads (Miltenyi Biotec) or with T Cell TransAct™ polymeric nanomatrix (Miltenyi Biotec) at the indicated bead:cell or volume:volume ratios, respectively. The percentage of T_{SCM} and T_{CM} cells at Day 14 was determined by flow cytometry. 561=donor number; 658=donor number; UT=non-transduced T cells; M=T Cell Activation/Expansion Kit; T=TransAct™; FMC=FMC63-41BB-CD3ζ anti-CD19 CAR T cells; D14=Day 14; T_{EFF}=effector T cells; T_{EM}=effector memory T cells; T_{CM}=central memory T cells; T_{SCM}=stem cell memory T cells.

### DETAILED DESCRIPTION

The present disclosure relates to methods for preparing T cells for use in a T cell therapy. In particular, the present disclosure relates to enriching the percentage of certain T cell subtypes, including immature, less differentiated T cells (e.g. stem memory T cells (hereinafter “T_{SCM}”) in a T cell population. By enriching for immature, less differentiated T cells, the potential persistence of T cells once administered to a subject, e.g., a patient may be increased. As a result, the enriched population of immature T cells is more likely to generate a sustained anti-tumor effect than a population of T cells at mixed stages of differentiation.

### Definitions

In order that the present disclosure can be more readily understood, certain terms are first defined. As used in this application, except as otherwise expressly provided herein, each of the following terms shall have the meaning set forth below. Additional definitions are set forth throughout the disclosure.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviations, per the practice in the art. Alternatively, “about” or “comprising essentially” can mean a range of up to 10% (i.e., +/−10%). For example, “about 3 mg” can include any number between 2.7 mg and 3.3 mg (for 10%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “activation” or “activated” refers to the state of an immune cell, e.g., a T cell, that has been sufficiently stimulated to induce detectable cellular proliferation.

Activation can also be associated with induced cytokine production and detectable effector functions. The term “activated T cells” refers to, among other things, T cells that are undergoing cell division. T cell activation can be characterized by increased T cell expression of one or more biomarker, including, but not limited to, CD57, PD1, CD107a, CD25, CD137, CD69, and/or CD71.

“Administering” refers to the physical introduction of an agent to a subject, using any of the various methods and delivery systems known to those skilled in the art. Exemplary routes of administration for the T cells prepared by the methods disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods.

The term “antibody” (Ab) includes, without limitation, an immunoglobulin which binds specifically to an antigen. In general, an antibody can comprise at least two heavy (H) chains and two light (L) chains interconnected by disulfide bonds. Each H chain comprises a heavy chain variable region (abbreviated herein as VH) and a heavy chain constant region. The heavy chain constant region can comprise three or four constant domains, CH1, CH2 CH3, and/or CH4. Each light chain comprises a light chain variable region (abbreviated herein as VL) and a light chain constant region. The light chain constant region can comprise one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs, including camelid antibodies. A nonhuman Ab can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “antigen binding molecule” or “antibody fragment” refers to any portion of an antibody less than the whole. An antigen binding molecule can include the antigenic complementarity determining regions (CDRs). Examples of antibody fragments include, but are not limited to, Fab, Fab′, F(ab′)₂, and Fv fragments, dAb, linear antibodies, scFv antibodies, and multispecific antibodies formed from antigen binding molecules.

The term “autologous” refers to any material derived from the same individual to which it is later to be re-introduced. For example, engineered autologous cell therapy (eACT™) involves collection of lymphocytes from a donor, e.g., a patient, which are then engineered to express, e.g., a CAR construct, and then administered back to the same donor, e.g., a patient.

The term “allogeneic” refers to any material derived from one individual which is then introduced to another individual of the same species, e.g., allogeneic T cell transplantation or therapy.

The term “anti-CD3/28 nanomatrix” refers to a nanometer scale matrix that comprises antibodies and/or fragments thereof that bind CD3 and CD 28 as provided by, e.g., Miltenyi Biotec Inc (Auburn, Calif.) as TransAct™ T Cell Reagent (see e.g. catalog number 200-076-202 MACS GMP T Cell Transact-CRR, catalog number 170-076-156 MACS GMP T Cell Transact for Research use).

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor at various stages. In certain embodiments, the cancer or tumor is stage 0, such that, e.g., the cancer or tumor is very early in development and has not metastasized. In some embodiments, the cancer or tumor is stage I, such that, e.g., the cancer or tumor is relatively small in size, has not spread into nearby tissue, and has not metastasized. In other embodiments, the cancer or tumor is stage II or stage III, such that, e.g., the cancer or tumor is larger than in stage 0 or stage I, and it has grown into neighboring tissues, but it has not metastasized, except potentially to the lymph nodes. In other embodiments, the cancer or tumor is stage IV, such that, e.g., the cancer or tumor has metastasized. Stage IV can also be referred to as advanced or metastatic cancer.

An “anti-tumor effect” as used herein, refers to a biological effect that can present as a decrease in tumor volume, an inhibition of tumor growth, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

The term “progression-free survival,” which can be abbreviated as PFS, as used herein refers to the time from the treatment date to the date of disease progression per the revised IWG Response Criteria for Malignant Lymphoma or death from any cause.

“Disease progression” is assessed by measurement of malignant lesions on radiographs or by other methods.

The “duration of response,” which can be abbreviated as DOR, as used herein refers to the period of time between a subject's first objective response to the date of confirmed disease progression, per the revised IWG Response Criteria for Malignant Lymphoma, or death.

The term “overall survival,” which can be abbreviated as OS, is defined as the time from the date of treatment to the date of death.

A “cytokine,” as used herein, refers to a non-antibody protein that can be released by immune cells, including macrophages, B cells, T cells, and mast cells to propagate an immune response. In some embodiments, one or more cytokines are released in response to the T cell therapy. In certain embodiments, those cytokines secreted in response to the T cell therapy can be a sign of effective T cell therapy.

“Therapeutically effective amount” or “therapeutically effective dosage,” as used herein, refers to an amount of the T cells or the DC cells that are produced by the present methods and that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of the T cells or DC cells to promote disease regression can be evaluated using a variety of methods known to the skilled practitioner, such as in human subjects during clinical trials, in animal model systems predictive of efficacy in humans, or by assaying the activity of the agent in in vitro assays.

The term “lymphocyte” as used herein can include natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation.

There are several types of T-cells, namely: Helper T-cells (e.g., CD4⁺ cells, effector T_{EFF} cells), Cytotoxic T-cells (also known as TC, cytotoxic T lymphocyte, CTL, T-killer cell, cytolytic T cell, CD8⁺ T-cells or killer T cell), Memory T-cells ((i) stem memory T_{SCM} cells, like naive cells, are CD45RO⁻, CCR7⁺, CD45RA⁺, CD62L⁺ (L-selectin), CD27⁺, CD28⁺ and IL-7Rα⁺, but they also express large amounts of CD95, IL-2Rβ, CXCR3, and LFA-1, and show numerous functional attributes distinctive of memory cells); (ii) central memory T_{CM} cells express L-selectin and are CCR7⁺ and CD45RO⁺ and they secrete IL-2, but not IFNβ or IL-4, and (iii) effector memory T_{EM} cells, however, do not express L-selectin or CCR7 but do express CD45RO and produce effector cytokines like IFNγ and IL-4), Regulatory T-cells (Tregs, suppressor T cells, or CD4⁺CD25⁺ regulatory T cells), Natural Killer T cells (NKT), and Gamma Delta T cells. T cells found within tumors are referred to as “tumor infiltrating lymphocytes” or “TILs.”

A “naive” T cell refers to a mature T cell that remains immunologically undifferentiated. Following positive and negative selection in the thymus, T cells emerge as either CD4⁺ or CD8⁺ naive T cells. In their naive state, T cells express L-selectin (CD62L⁺), IL-7 receptor-α (IL-7R-α), and CD132, but they do not express CD25, CD44, CD69, or CD45RO. As used herein, “immature” can also refer to a T cell which exhibits a phenotype characteristic of either a naive T cell or an immature T cell, such as a T_{SCM} cell or a T_{CM} cell. For example, an immature T cell can express one or more of L-selectin (CD62L⁺), IL-7R-α, CD132, CCR7, CD45RA, CD45RO, CD27, CD28, CD95, CXCR3, and LFA-1. Naive or immature T cells can be contrasted with terminal differentiated effector T cells, such as T_{EM} cells and T_{EFF} cells.

“T cell function,” as referred to herein, refers to normal characteristics of healthy T cells. In some embodiments, a T cell function comprises T cell proliferation. In some embodiments, a T cell function comprises a T cell activity. In some embodiments, the T cell function comprises cytolytic activity.

“Cell proliferation,” as used herein, refers to the ability of T cells to grow in numbers through cell division. Proliferation can be measured, e.g., by staining cells with carboxyfluorescein succinimidyl ester (CFSE). Cell proliferation can occur in vitro, e.g., during T cell culture, or in vivo, e.g., following administration of a T cell therapy.

“T cell activity,” as used herein, refers to any activity common to healthy T cells. In some embodiments, the T cell activity comprises cytokine production. In certain embodiments, the T cell activity comprises production of one or more cytokine selected from interferon gamma (IFNγ), tissue necrosis factor alpha (TNFα), and both.

A “cytolytic activity” or “cytotoxicity,” as used herein, refers to the ability of a T cell to destroy a target cell. In some embodiments, the target cell is a cancer cell, e.g., a tumor cell. In some embodiments, the T cell expresses a chimeric antigen receptor (CAR) or a T cell receptor (TCR), and the target cell expresses a target antigen.

The term “genetically engineered,” “gene editing,” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which can either be obtained from a patient or a donor. The cell can be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy can include adoptive T cell therapy, tumor-infiltrating lymphocyte (TIL) immunotherapy, autologous cell therapy, engineered autologous cell therapy (eACT™), engineered allogeneic cell therapy, and allogeneic T cell transplantation. However, one of skill in the art will recognize that the methods of preparing T cells disclosed herein would enhance the effectiveness of any transplanted T cell therapy. Examples of T cell therapies are described in U.S. Patent Publication Nos. 2014/0154228 and 2002/0006409, and International Publication No. WO 2008/081035.

The T cells of the immunotherapy can come from any source known in the art. For example, T cells can be differentiated in vitro from a hematopoietic stem cell population, or T cells can be obtained from a donor. The donor can be a subject, e.g., a subject in need of an anti-cancer treatment, or a healthy donor. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. T cells can also be obtained from an artificial thymic organoid (ATO) cell culture system, which replicates the human thymic environment to support efficient ex vivo differentiation of T-cells from primary and reprogrammed pluripotent stem cells. Additional methods of isolating T cells for a T cell therapy are disclosed in U.S. Patent Publication No. 2013/0287748, which is herein incorporated by references in its entirety.

The term “engineered Autologous Cell Therapy,” which can be abbreviated as “eACT™” also known as adoptive cell transfer, is a process by which a patient's own T cells are collected and subsequently genetically altered to recognize and target one or more antigens expressed on the cell surface of one or more specific tumor cells or malignancies. T cells can be engineered to express, for example, one or more chimeric antigen receptors (CAR) or one or more T cell receptor (TCR) and combinations thereof. CAR positive (+) T cells are engineered to express an extracellular single chain variable fragment (scFv) with specificity for a particular tumor antigen linked to an intracellular signaling part comprising a costimulatory domain and an activating domain. The costimulatory domain can be derived from, e.g., CD28, CTLA4, CD16, OX-40, 4-1BB/CD137, CD2, CD7, CD27, CD30, CD40, programmed death-1 (PD-1), programmed death ligand-1 (PD-L1), inducible T cell costimulator (ICOS), ICOS-L, lymphocyte function-associated antigen-1 (LFA-1 (CD11a/CD18), CD3 gamma, CD3 delta, CD3 epsilon, CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof; The activating domain can be derived from, e.g., CD3, such as CD3 zeta, epsilon, delta, gamma, or the like. In certain embodiments, the CAR is designed to have two, three, four, or more costimulatory domains. The CAR scFv can be designed to target, for example, CD19, which is a transmembrane protein expressed by cells in the B cell lineage, including all normal B cells and B cell malignances, including but not limited to NHL, CLL, and non-T cell ALL. Example CAR+ T cell therapies and constructs are described in U.S. Patent Publication Nos. 2013/0287748, 2014/0227237, 2014/0099309, and 2014/0050708, and these references are incorporated by reference in their entirety.

A “patient” as used herein includes any human who is afflicted with a disease, including cancer (e.g., a lymphoma or a leukemia). The terms “subject” and “patient” are used interchangeably herein. The term “donor subject” refers to herein a subject whose cells are being obtained for further in vitro engineering. The donor subject can be a cancer patient that is to be treated with a population of cells generated by the methods described herein (i.e., an autologous donor), or can be an individual who donates a lymphocyte sample that, upon generation of the population of cells generated by the methods described herein, will be used to treat a different individual or cancer patient (i.e., an allogeneic donor). Those subjects who receive the cells that were prepared by the present methods can be referred to as a “recipient subject.”

“Stimulation,” as used herein, refers to a primary response induced by binding of a stimulatory molecule with its cognate ligand, wherein the binding mediates a signal transduction event. A “stimulatory molecule” is a molecule on a T cell, e.g., the T cell receptor (TCR)/CD3 complex, that specifically binds with a cognate stimulatory ligand present on an antigen present cell. A “stimulatory ligand” is a ligand that when present on an antigen presenting cell (e.g., an artificial antigen presenting cell (aAPC), a dendritic cell, a B-cell, and the like) can specifically bind with a stimulatory molecule on a T cell, thereby mediating a primary response by the T cell, including, but not limited to, activation, initiation of an immune response, proliferation, and the like. Stimulatory ligands include, but are not limited to, an MHC Class I molecule loaded with a peptide, an anti-CD3 antibody, a superagonist anti-CD28 antibody, and a superagonist anti-CD2 antibody. An “activated” or “active,” as used herein, refers to a T cell that has been stimulated. An active T cell can be characterized by expression of one or more marker selected from the group consisting of CD137, CD25, CD71, CD26, CD27, CD28, CD30, CD154, CD4OL, and CD134.

The term “exogenous” refers to any substance derived from an external source.

The term “persistence,” as used herein, refers to the ability of, e.g., one or more transplanted T cells administered to a subject or their progenies (e.g., differentiated or matured T cells) to remain in the subject at a detectable level for a period of time. As used herein, increasing the persistence of one or more transplanted T cells or their progenies (e.g., differentiated or matured T cells) refers to increasing the amount of time the transplanted T cells are detectable in a subject after administration. For example, the in vivo persistence of one or more transplanted T cells can be increased by at least about at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 3 weeks, at least about 4 weeks, at least about 1 month, at least about 2 months, at least about 3 months, at least about 4 months, at least about 5 months, or at least about 6 months. In addition, the in vivo persistence of one or more transplanted T cells can be increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold compared to the one or more transplanted T cells that were not prepared by the present methods disclosed herein.

The terms “reducing” and “decreasing” are used interchangeably herein and indicate any change that is less than the original value. “Reducing” and “decreasing” are relative terms, requiring a comparison between pre- and post-measurements. “Reducing” and “decreasing” include complete depletions. In some embodiments, the terms “reducing” and “decreasing” include a comparison of T cell effects between the T cells prepared by the presently disclosed methods (e.g., strong activation) and the T cells without the preparation.

The term “modulating” T cell maturation, as used herein, refers to the use of any intervention described herein to influence the maturation, e.g. differentiation, of one or more T cells. In some embodiments, “modulating” refers to delaying or inhibiting T cell maturation. In other embodiments, “modulating” refers to accelerating or promoting T cell maturation. In particular, “delaying or inhibiting T cell maturation,” as used here, refers to maintaining one or more T cells in an immature or undifferentiated state. For example, “delaying or inhibiting T cell maturation” can refer to maintaining T cells in a naive or TCM state, as opposed to progressing to a T_{EM} or T_{EFF} state. “Delaying or inhibiting T cell maturation” can also refer to increasing or enriching the overall percentage of immature or undifferentiated T cells (e.g., naive T cells and/or T_{CM} cells) within a mixed population of T cells. The state of a T cell (e.g., as mature or immature) can be determined, e.g., by screening for the expression of various genes and the presence of various proteins expressed on the surface of the T cells. For example, the presence of one or more marker selected from the group consisting of L-selectin (CD62L⁺), IL-7R-a, CD132, CR7, CD45RA, CD45RO, CD27, CD28, CD95, IL-2Rβ, CXCR3, LFA-1, and any combination thereof can be indicative of less mature, undifferentiated T cells.

Treatment” or “treating” of a subject refers to any type of intervention or process performed on, or the administration of one or more T cells prepared using the present disclosure, to the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission.

Various aspects of the disclosure are described in further detail in the following subsections.

### Methods of Preparing Immune Cells

The present disclosure relates to methods for preparing immune cells (e.g., lymphocytes or dendritic cells) for use in a cell therapy. It is found that certain in vitro engineered cells (e.g., CAR T cells, T cells, or dendritic cells) may not be as effective when administered to a patient after in vitro engineering. Without intending to be bound by any particular theory, it is noted that one reason can be that lymphocytes can be prematurely differentiated in vitro before being administered to a patient. The present disclosure, in certain embodiments, sets forth methods to enrich the number of immature, less differentiated cells in a T cell population by using strong activation conditions.

In one embodiment, the present disclosure relates to methods of increasing the percentage of immature, less differentiated cells (e.g., stem cell memory T cells; T_{SCM}) in the population of collected T cells. Accordingly, the methods described herein can be used to increase the in vivo persistence of transplanted T cells or DC cells or their progenies in a cell therapy (e.g., T cell therapy). In addition, in some embodiments the present invention provides that the resulting T cells exhibit increased expansion in vitro. Further, in some embodiments the present invention provides that the resulting T cells exhibit an increased percentage of CD8+ T cells.

In another embodiment, the invention includes methods for enriching the percentage of immature less differentiated cells in a T cell population by contacting one or more cells with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population where in the T cell population comprises a cell density of about 0.2×10⁶ cells/ml to about 5.8×10⁶ cells/ml. Further preparation of the T cells is described elsewhere herein.

The present disclosure demonstrates that contacting a cell population comprising one or more T cells in vitro with a higher concentration of anti-CD3/28 nanomatrix can increase the concentration of immature and less differentiated T cells, e.g. T_{SCM} cells in a sample, relative to the concentration of more terminally differentiated T cells. Accordingly, in another embodiment, the present disclosure provides a method for generating stem cell-like CD4⁺ T cells or CD8⁺ T cells comprising culturing one or more T cells in a medium comprising a ratio of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of a T cell population where in the T cell population comprises a cell density of about 0.2×10⁶ cells/ml to about 5.8×10⁶ cells/ml. In other embodiments, the present disclosure provides method of enriching a population of CD8⁺ CD45RA⁺/CD62L⁺ T cells and/or CD8⁺ CD45RO⁻/CD62L⁺ T cells in a sample comprising (a) contacting the one or more T cells with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population where in the T cell population comprises a cell density of about 0.2×10⁶ cells/ml to about 5.8×10⁶ cells/ml and (c) expanding the one or more T cells. Generating an increased concentration of immature and undifferentiated T cells or DC cells can increase the in vivo persistence of the cells upon transplantation to a subject in need of a cell therapy (e.g., T cell therapy or DC cell therapy). Thus, in another embodiment, the present disclosure provides a method for extending the in vivo persistence of one or more T cells in an adoptive cell therapy comprising contacting the one or more T cells with (i) with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population where in the T cell population comprises a cell density of about 0.2×10⁶ cells/ml to about 5.8×10⁶cells/m1 prior to administration to a subject; wherein the in vivo persistence is extended relative to one or more transplanted T cells contacted with one volume of anti-CD3/28 nanomatrix, e.g. T Cell Transact™ to more than 15 volumes (e.g. 16, 16.5, 17, 17.5, 18, 18.5, 19, 19.5, 20, 25, 30 or 40 volumes) of the T cell population.

The methods disclosed herein comprise modulating, e.g., enriching a T cell population for immature, less differentiated cells in vitro. The delay or inhibition of the maturation or differentiation of the one or more T cells or DC cells can be measured by any methods known in the art. For example, the enrichment of a cell population for immature, less differentiated T cells or DC cells can be measured by detecting the presence of one or biomarkers. The presence of the one or more biomarkers can be detected by any method known in the art, including, but not limited to, immunohistochemistry and/or fluorescence-activated cell sorting (FACS). In some embodiments, the one or more biomarkers is selected from the group consisting of L-selectin (CD62L⁺), IL-7Rα, CD132, CCR7, CD45RA, CD45RO, CD27, CD28, CD95, IL-2Rβ, CXCR3, LFA-1, or any combination thereof. In certain embodiments, the enrichment of a cell population for immature, less differentiated T cells can be measured by detecting the presence of one or more of L-selectin (CD62L⁺), CD45RA and CD45RO. One of skill in the art will appreciate that although the present methods can increase the relative proportion of immature and undifferentiated T cells or DC cells in a population of collected cells, some mature and differentiated cells can still be present. As a result, the enrichment of a cell population for immature, less differentiated T cells can be measured by calculating the total percent of immature and undifferentiated cells in a cell population before and after contacting one or more cells with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population where in the T cell population comprises a cell density of about 0.2×10⁶ cells/ml to about 5.8×10⁶ cells/ml. methods disclosed herein increase the percentage of immature and undifferentiated T cells in a T cell population. In certain embodiments, the one or more T cells contacted with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population comprise at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% immature or undifferentiated T cells. In other embodiments, the one or more T cells or contacted with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population where in the T cell population comprises a cell density of about 0.2×10⁶ cells/ml to about 5.8×10⁶ cells/ml comprise at least about 10% to at least about 90%, at least about 20% to at least about 80%, at least about 30% to at least about 70%, at least about 40% to at least about 60%, at least about 10% to at least about 50%, at least about 20%, at least about 40%, at least about 35% to at least about 45%, at least about 20% to at least about 60%, or at least about 50% to at least about 90% immature or undifferentiated T cells. In certain embodiments, the immature or undifferentiated T cells are naive T cells and/or central memory T_{SCM} cells.

In another embodiment the enrichment of a cell population for immature, less differentiated T cells or DC cells can be measured by calculating the total percent of immature and undifferentiated cells in a cell population that has been contacted with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population where in the T cell population comprises a cell density of about 0.2×10⁶ cells/ml to about 5.8×10⁶ cells/ml and comparing to the percentage of immature, less differentiated T cells or DC cells in a reference cell population that has been contacted with one volume of anti-CD3/28 nanomatrix, e.g. T Cell Transact™ to 1 more than 17 volumes of the T cell population where in the reference T cell population comprises a cell density of about 0.2×10⁶ cells/ml to about 5.8×10⁶ cells/ml. In certain embodiments, the one or more T cells contacted with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes)of the T cell population comprise at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% more immature or undifferentiated T cells, e.g. T_{SCM} cells than the reference T Cell population. In certain embodiments, the one or more T cells contacted with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes)of the T cell population comprise 2 fold, 3 fold, 4 fold, 5 fold, 6 fold 7 fold, 8 fold, 9 fold or 10 fold a more immature or undifferentiated T cells, e.g. T_{SCM} cells than the reference T Cell population.

The present disclosure provides a method for enriching the percentage of immature less differentiated cells in a T cell population by contacting one or more T cells with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population where in the T cell population comprises a cell density of about 0.2×10⁶ cells/ml to about 5.8×10⁶ cells/ml. The one or more T cells or DC cells can be contacted the anti-CD3/28 nanomatrix through any means known in the art. For example, the anti-CD3/28 nanomatrix can be added to a culture medium used to culture the one or more T cells or DC cells.

The one or more T cells of the present disclosure can be administered to a subject for use in a T cell therapy. Accordingly, the one or more T cells can be collected from a subject in need of a T cell therapy or from a donor. Once collected, the one or more T cells can be processed for any suitable period of time before being administered to a subject. During this time, the one or more T cells can be contacted with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population where in the T cell population comprises a cell density of about 0.2×10⁶ cells/ml to about 5.8×10⁶ cells/ml for any period of time between the collection of the T cells from the donor and the administration of a subject. For example, the one or more T cells can be contacted with, e.g., cultured in the presence with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days or at least about 20 days. In some embodiments, the one or more T cells are contacted with, (e.g., cultured in the presence of) with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population for about 1 day to about 18 days, for about 1 day to about 14, for about 1 day to about 10 days, for about 1 day to about 7 days, from about 1 day to about 6 days, from about 1 day to about 5 days, from about 1 day to about 4 days, from about 1 day to about 3 days, from about 1 day to about 2 days, from about 2 days to about 3 days, from about 2 days to about 4 days, from about 2 days to about 5 days, or from about 2 days to about 6 days. In one particular embodiment, the one or more T cells are contacted with, e.g., cultured in the presence of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population from the day the T cells are collected (e.g., day-0) until the day the T cells are administered to a subject. In another embodiment, the T cells are contacted with, e.g., cultured in the presence of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population from day 0 to administration, from day 1 to administration, from day 2 to administration, from day 3 to administration from day 4 to administration, from day 5 to administration, or from day 6 to administration. In some embodiments, the one or more T cells are washed prior to administration to remove the anti-CD3/28 nanomatrix.

The methods described herein can further comprise enriching a population of lymphocytes obtained from a donor. Enrichment of a population of lymphocytes, e.g., the one or more T cells, can be accomplished by any suitable separation method including, but not limited to, the use of a separation medium (e.g., FICOLL®PAQUE, ROSETTESEP™ HLA Total Lymphocyte enrichment cocktail, Lymphocyte Separation Medium (LSA) (MP Biomedical Cat. No. 0850494X), or the like), cell size, shape or density separation by filtration or elutriation, immunomagnetic separation (e.g., magnetic-activated cell sorting system, MACS), fluorescent separation (e.g., fluorescence activated cell sorting system, FACS), or bead-based column separation. Additionally, enrichment of a population of T cells can be accomplished using a Pan T Isolation kit, human (Miltenyi Biotec Inc., Auburn, Calif., #130-096-535), CD4/CD8 selection kit (Miltenyi Biotec Inc., Auburn, Calif., #130-122-352 StraightFrom® Leukopak® CD4/CD8 MicroBead Kit, #130-030-401 CliniMACS CD4 MicroBeads and #130-030-801 CliniMACS CD8 MicroBeads and CD3 Microbead Kit).

The methods described herein can further comprise stimulating the population of lymphocytes with one or more T cell stimulating agents to produce a population of activated T cells under a suitable condition. The lymphocytes e.g., T cells or PBMCs, can be fresh or frozen, they can be derived from for example, peripheral blood or umbilical cord blood. Any combination of one or more suitable T cell stimulating agents can be used to produce a population of activated T cells including, including, but not limited to, an antibody or functional fragment thereof which targets a T cell stimulatory or co-stimulatory molecule (e.g., anti-CD2 antibody, anti-CD3 antibody, anti-CD28 antibody, or a functional fragment thereof), or any other suitable mitogen (e.g., tetradecanoyl phorbol acetate (TPA), phytohaemagglutinin (PHA), concanavalin A (conA), lipopolysaccharide (LPS), pokeweed mitogen (PWM)), or a natural ligand to a T-cell stimulatory or co-stimulatory molecule or an anti-CD3/28 nanomatrix, e.g. TransAct™ T Cell Reagent (Miltenyi Biotec Inc., Auburn, Calif.).

The suitable condition for stimulating the population of lymphocytes as described herein can include a temperature, for an amount of time, and/or in the presence of a level of CO₂. In certain embodiments, the temperature for stimulation is about 34° C., about 35° C., about 36° C., about 37° C., or about 38° C. In certain embodiments, the temperature for stimulation is about 34-38° C. In certain embodiments, the temperature for stimulation is from about 35-37° C. In certain embodiments, the temperature for stimulation is from about 36-38° C. In certain embodiments, the temperature for stimulation is about 36-37° C. or about 37° C.

Another condition for stimulating the population of lymphocytes as described herein can include a time for stimulation. In some embodiments, the time for stimulation is about 0 to 72 hours. In some embodiments, the time for stimulation is about 0-24 hours, about 24-72 hours, about 24-36 hours, about 30-42 hours, about 36-48 hours, about 40-52 hours, about 42-54 hours, about 44-56 hours, about 46-58 hours, about 48-60 hours, about 54-66 hours, or about 60-72 hours. In one particular embodiment, the time for stimulation is about 48 hours or at least about 48 hours. In other embodiments, the time for stimulation is about 44-52 hours. In certain embodiments, the time for stimulation is about 40-44 hours, about 40-48 hours, about 40-52 hours, or about 40-56 hours. In one embodiment the time for stimulation is about 0-24 hours and the population of lymphocytes is fresh.

Other conditions for stimulating the population of lymphocytes as described herein can include a CO₂ Level. In some embodiments, the level of CO₂ for stimulation is about 1.0-10% CO₂. In some embodiments, the level of CO₂ for stimulation is about 1.0%, about 2.0%, about 3.0%, about 4.0%, about 5.0%, about 6.0%, about 7.0%, about 8.0%, about 9.0%, or about 10.0% CO₂. In one embodiment, the level of CO₂ for stimulation is about 3-7% CO₂. In other embodiments, the level of CO₂ for stimulation is about 4-6% CO₂. In still other embodiments, the level of CO₂ for stimulation is about 4.5-5.5% CO₂. In one particular embodiment, the level of CO₂ for stimulation is about 5% CO₂.

The conditions for stimulating the population of lymphocytes can comprise a temperature, for an amount of time for stimulation, and/or in the presence of a level of CO₂ in any combination. For example, the step of stimulating the population of lymphocytes can comprise stimulating the population of lymphocytes with one or more T-cell stimulating agents at a temperature of about 36-38° C., for an amount of time of about 44-52 hours, and in the presence of a level of CO₂ of about 4.5-5.5% CO₂.

The concentration of lymphocytes useful for the methods herein can be about 0.5-10.0×10⁶ cells/mL. In certain embodiments, the concentration of lymphocytes is about 1.0-2.0×10⁶ cells/mL, about 1.0-3.0×10⁶ cells/mL, about 1.0-4.0×10⁶ cells/mL, about 1.0-5.0×10⁶ cells/mL, about 1.0-6.0×10⁶ cells/mL, about 1.0-7.0×10⁶ cells/mL, about 1.0-8.0×10⁶ cells/mL, 1.0-9.0×10⁶ cells/mL or about 1.0-10.0×10⁶ cells/mL. In certain embodiments, the concentration of lymphocytes is about 1.0-2.0×10⁶ cells/mL or 2.0-3.0.×10⁶ cells/mL. In certain embodiments, the concentration of lymphocytes is about 1.0-1.2×10⁶ cells/mL, about 1.0-1.4×10⁶ cells/mL, about 1.0-1.6×10⁶ cells/mL, about 1.0-1.8×10⁶ cells/mL, or about 1.0-2.0×10⁶ cells/mL. In certain embodiments, the concentration of lymphocytes is about 2.1-3.0×10⁶ cells/mL, about 2.1-3.0×10⁶ cells/mL, about 2.1-3.0×10⁶ cells/mL, about 2.4-3×10⁶ cells/mL, about 2.5-3.0×10⁶ cells/mL, about 2.6-3.0×10⁶ cells/mL, about 2.7-3.0×10⁶ cells/mL, 2.8-3.0×10⁶ cells/mL or 2.8-3.0×10⁶ cells/mL. In certain embodiments, the concentration of lymphocytes is at least about 0.2×10⁶ cells/mL, 0.3×10⁶ cells/mL, 0.4×10⁶ cells/mL, 0.5×10⁶ cells/mL, 0.6×10⁶ cells/mL, 0.7×10⁶ cells/mL, 0.8×10⁶ cells/mL, 0.9×10⁶ cells/mL, 1.0×10⁶ cells/mL, at least about 1.1×10⁶ cells/mL, at least about 1.2×10⁶ cells/mL, at least about 1.3.×10⁶ cells/mL cells/mL, at least about 1.4×10⁶ cells/mL, at least about 1.5.×10⁶ cells/mL, at least about 1.6.×10⁶ cells/mL, at least about 1.7.×10⁶ cells/mL, at least about 1.8×10⁶ cells/mL, at least about 1.9.×10⁶ cells/mL, at least about 2.0.×10⁶ cells/mL, at least about 4.0×10⁶ cells/mL, at least about 6.0×10⁶ cells/mL, at least about 8.0×10⁶, or at least about 10.0×10⁶ cells/mL.

An anti-CD3 antibody (or functional fragment thereof), an anti-CD28 antibody (or functional fragment thereof), or a combination of anti-CD3 and anti-CD28 antibodies can be used in accordance with the step of stimulating the population of lymphocytes. Any soluble or immobilized anti-CD2, anti-CD3 and/or anti-CD28 antibody or functional fragment thereof can be used (e.g., clone OKT3 (anti-CD3), clone 145-2C11 (anti-CD3), clone UCHT1 (anti-CD3), clone L293 (anti-CD28), clone 15E8 (anti-CD28)). In some aspects, the antibodies can be purchased commercially from vendors known in the art including, but not limited to, Miltenyi Biotec (e.g. TransAct™ T Cell Reagent Large Scale, #130-109-104), BD Biosciences (e.g., MACS GMP CD3 pure 1 mg/mL, Part No. 170-076-116), and eBioscience, Inc. Further, one skilled in the art would understand how to produce an anti-CD3 and/or anti-CD28 antibody by standard methods. In some embodiments, the one or more T cell stimulating agents that are used in accordance with the step of stimulating the population of lymphocytes include an antibody or functional fragment thereof which targets a T-cell stimulatory or co-stimulatory molecule in the presence of a T cell cytokine. In one aspect, the one or more T cell stimulating agents include an anti-CD3 antibody and IL-2. In certain embodiments, the T cell stimulating agent includes an anti-CD3 antibody at a concentration of from about 20 ng/mL-100 ng/mL. In certain embodiments, the concentration of anti-CD3 antibody is about 20 ng/mL, about 30 ng/mL, about 40 ng/mL, about 50 ng/mL, about 60 ng/mL, about 70 ng/mL, about 80 ng/mL, about 90 ng/mL, or about 100 ng/mL. In one particular embodiment, the concentration of anti-CD3 antibody is about 50 ng/mL.

The methods described herein can further comprise transducing the population of activated T cells with a viral vector comprising a nucleic acid molecule which encodes a protein of interest (e.g., a CAR), using a single cycle transduction to produce a population of transduced T cells. Several recombinant viruses have been used as viral vectors to deliver genetic material to a cell. Viral vectors that can be used in accordance with the transduction step can be any ecotropic or amphotropic viral vector including, but not limited to, recombinant retroviral vectors, recombinant lentiviral vectors, recombinant adenoviral vectors, and recombinant adeno-associated viral (AAV) vectors. In some embodiments, the method further comprises transducing the one or more T cells with a retrovirus. In one embodiment, the viral vector used to transduce the population of activated T cells is an MSGV1 gamma retroviral vector. According to one aspect of this embodiment, the viral vector is grown in a suspension culture in a medium which is specific for viral vector manufacturing referred to herein as a “viral vector inoculum.” Any suitable growth media and/or supplements for growing viral vectors can be used in the viral vector inoculum in accordance with the methods provided herein. According to some aspects, the viral vector inoculum is added to culture media during the transduction step.

In some embodiments, the one or more T cells can be transduced with a lentivirus. In one embodiment, the lentivirus comprises a heterologous gene encoding a protein of interest. In one particular embodiment, the protein of interest is capable of binding an antigen on the surface of a target cell, e.g., on the surface of a tumor cell, and can be a CAR.

The conditions for transducing the population of activated T cells as described herein can comprise a specific time, at a specific temperature and/or in the presence of a specific level of CO₂. In certain embodiments, the temperature for transduction is about 34° C., about 35° C., about 36 C, about 37° C., or about 38° C. In one embodiment, the temperature for transduction is about 34-38° C. In another embodiment, the temperature for transduction is from about 35-37° C. In another embodiment, the temperature for transduction is from about 36-38° C. In still another embodiment, the temperature for transduction is about 36-37° C. In one particular embodiment, the temperature for transduction is about 37° C.

In certain embodiments, the time for transduction is about 0-36 hours after activation. In some embodiments, the time for transduction is about 12-16 hours, about 12-20 hours, about 12-24 hours, about 12-28 hours, or about 12-32 hours. In other embodiments, the time for transduction is about 20 hours or at least about 20 hours. In one embodiment, the time for transduction is about 16-24 hours. In other embodiments, the time for transduction is at least about 14 hours, at least about 16 hours, at least about 18 hours, at least about 20 hours, at least about 22 hours, at least about 24 hours, or at least about 26 hours.

In certain embodiments, the level of CO₂ for transduction is about 1.0-10% CO₂. In other embodiments, the level of CO₂ for transduction is about 1.0%, about 2.0%, about 3.0%, about 4.0%, about 5.0%, about 6.0%, about 7.0%, about 8.0%, about 9.0%, or about 10.0% CO₂. In one embodiment, the level of CO₂ for transduction is about 3-7% CO₂. In another embodiment, the level of CO₂ for transduction can be about 4-6% CO₂. In another embodiment, the level of CO₂ for transduction is about 4.5-5.5% CO₂. In one particular embodiment, the level of CO₂ for transduction is about 5% CO₂.

In some embodiments, transducing the population of activated T cells as described herein can be performed for a particular time, at a specific temperature and/or in the presence of a specific level of CO₂ in any combination: a temperature of about 36-38° C., for an amount of time of about 16-24 hours, and in the presence of a level of CO₂ of about 4.5-5.5% CO₂.

The methods provided herein can comprise expanding the population of transduced one or more T cells for a particular time, in order to produce a population of engineered T cells. The predetermined time for expansion can be any suitable time which allows for the production of (i) a sufficient number of cells in the population of engineered T cells for at least one dose for administering to a patient, (ii) a population of engineered T cells with a favorable proportion of juvenile cells compared to a typical longer process, or (iii) both (i) and (ii). This time will depend on the protein of interest expressed by the T cells, the vector used, the dose that is needed to have a therapeutic effect, and other variables. Thus, in some embodiments, the predetermined time for expansion can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, or more than 21 days. In some aspects, the time for expansion is shorter than expansion methods known in the art. For example, the predetermined time for expansion can be shorter by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or can be shorter by more than 75%. In one aspect, the time for expansion is about 3 days, and the time from enrichment of the population of lymphocytes to producing the engineered T cells is about 6 days.

The conditions for expanding the population of transduced T cells can include a temperature and/or in the presence of a level of CO₂. In certain embodiments, the temperature is about 34° C., about 35° C., about 36° C., about 37° C., or about 38° C. In one embodiment, the temperature is about 34-38° C. In another embodiment, the temperature is from about 35-37° C. In another embodiment, the temperature is from about 36-38° C. In yet another embodiment, the temperature is about 36-37° C. In one particular embodiment the temperature is about 37° C. In certain embodiments, the level of CO₂is 1.0-10% CO₂. In other embodiments, the level of CO₂ is about 1.0%, about 2.0%, about 3.0%, about 4.0%, about 5.0%, about 6.0%, about 7.0%, about 8.0%, about 9.0%, or about 10.0% CO₂. In one embodiment, the level of CO₂ is about 4.5-5.5% CO₂. In another embodiment, the level of CO₂ is about 5% CO₂. In other embodiments, the level of CO₂ is about 3.5%, about 4.0%, about 4.5%, about 5.0%, about 5.5%, or about 6.5% CO₂. In some embodiments, the conditions for expanding the population of transduced T cells include a temperature and/or in the presence of a level of CO₂ in any combination. For example, conditions for expanding the population of transduced T cells comprise a temperature of about 36-38° C. and in the presence of a level of CO₂ of about 4.5-5.5% CO₂.

Each step of the methods provided herein can be performed in a semi-closed or a closed system. In certain embodiments, a closed system is a closed bag culture system, comprising any suitable cell culture bags (e.g., Miltenyi Biotec MACS® GMP Cell Differentiation Bags, Origen Biomedical PermaLife Cell Culture bags). In some embodiments, the cell culture bags used in the closed bag culture system are coated with a recombinant human fibronectin fragment during the transduction step. The recombinant human fibronectin fragment can include three functional domains: a central cell-binding domain, heparin-binding domain II, and a CS1-sequence. The recombinant human fibronectin fragment can be used to increase gene efficiency of retroviral transduction of immune cells by aiding co-localization of target cells and viral vector. In certain embodiments, the recombinant human fibronectin fragment is RETRONECTIN®. (Takara Bio, Japan). In certain embodiments, the cell culture bags are coated with recombinant human fibronectin fragment at a concentration of about 1-60 μg/mL or about 1-40 μg/mL. In other embodiments, the cell culture bags are coated with recombinant human fibronectin fragment at a concentration of about 1-20 μg/mL, 20-40 μg/mL, or 40-60 μg/mL. In some embodiments, the cell culture bags are coated with about 1 μg/mL, about 2μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, or about 20 μg/mL recombinant human fibronectin fragment. In other embodiments, the cell culture bags are coated with about 2-5 μg/mL, about 2-10 μg/mL, about 2-20 μg/mL, about 2-25 μg/mL, about 2-30 μg/mL, about 2-35 μg/mL, about 2-40 μg/mL, about 2-50 μg/mL, or about 2-60 μg/mL recombinant human fibronectin fragment. In certain embodiments, the cell culture bags are coated with at least about 2 μg/mL, at least about 5 μg/mL, at least about 10 μg/mL, at least about 15 μg/mL, at least about 20 μg/mL, at least about 25 μg/mL, at least about 30 μg/mL, at least about 40 μg/mL, at least about 50 μg/mL, or at least about 60 μg/mL recombinant human fibronectin fragment. In one particular embodiment, the cell culture bags are coated with at least about 10 μg/mL recombinant human fibronectin fragment. The cell culture bags used in the closed bag culture system can optionally be blocked with human albumin serum (HSA) during the transduction step. In an alternative embodiment, the cell culture bags are not blocked with HSA during the transduction step.

In other aspects, at least one of a) contacting one or more T cells with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population, (b) transducing the population of activated T cells, and (c) expanding the population of transduced T cells is performed using a T cell culture medium with or without human serum. In some aspect, each of (a) to (c) is performed using a T cell culture medium which is free from added serum. As referred to herein, the term “serum-free media” or “serum-free culture medium” means that the growth media used is not supplemented with serum (e.g., human serum or bovine serum). Thus, in some embodiments, no serum is added to the culture medium as an individually separate and distinct ingredient for the purpose of supporting the viability, activation and grown of the cultured cells. Any suitable culture medium T cell growth media can be used for culturing the cells in suspension in accordance with the methods described herein. For example, a T cell growth media can include, but is not limited to, a sterile, low glucose solution that includes a suitable amount of buffer, magnesium, calcium, sodium pyruvate, and sodium bicarbonate. In one embodiment, the T cell growth media is OPTMIZER™ (Life Technologies). In contrast to typical methods for producing engineered T cells, the methods described herein can use culture medium that is not supplemented with serum (e.g., human or bovine).

### Anti-CD3/28 Nanomatrix

The anti-CD3/28 nanomatrix of the instant disclosure are nanometer scale matrices comprising antibodies and/or fragments thereof that bind CD3 and CD 28 and are provided by Miltenyi Biotec Inc (Auburn, Calif.) as TransAct™ T Cell Reagent (hereinafter “TransAct™). The TransAct™ reagent is a colloidal reagent consisting of nanoscale iron oxide crystals embedded into a biocompatible polysaccharide matrix with an overall diameter of about 100 nm. Antibodies against CD3 (clone OKT3) and CD28 (clone 15E8) are covalently attached to the matrix. See, e.g. Casati et al., Cancer Immunol Immunother 2013 October; 62(10):1563-73, Casati et al., MACS & more, Vol 15, February 2013, and US2014/0087462.

### T Cells

The one or more T cells described herein can be obtained from any source, including, for example, a human donor. The donor can be a subject in need of an anti-cancer treatment, e.g., treatment with one or more T cells generated by the methods described herein (i.e., an autologous donor), or can be an individual that donates a lymphocyte sample that, upon generation of the population of cells generated by the methods described herein, will be used to treat a different individual or cancer patient (i.e., an allogeneic donor). The population of lymphocytes can be obtained from the donor by any suitable method used in the art.

The methods described herein can be used to increase the percentage of less differentiated, immature cells (e.g. T_{SCM} cells) in a T cell population by contacting one or more cells with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population. As described herein, it was surprisingly found that strong activation of one or more T cells with anti-CD3/28 nanomatrix, e.g. T Cell Transact™. increases the percentage of naive and immature T cells in vitro. In particular, following activation, the one or more T cells can express one or more genes indicative of undifferentiated or immature T cells. The one or more genes indicative of undifferentiated or immature T cells can be selected from the group CD8, CD45RA, CCR7, CD45RO, CD62L, CD28, CD95, IL-7Ra, CXCR4, TCF7, FOXO1, ID3, BCL6, and any combination thereof. For example, contacting one or more T cells with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population results in an increase in the percent of cells expressing one or more genes indicative of undifferentiated or immature T cells selected from CD62L, CD45RA, CD45RO, and any combination thereof.

In other embodiments, the one or more T cells express CD62L, CD45RA and/or CD45RO following contacting one or more T cells with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population In one particular embodiment, a greater percentage of the one or more T cells express CD62L, CD45RA and/or CD45RO after as compared to before being contacted with the antiCD3/28 nanomatrix. In one particular embodiment, a greater percentage of the one or more T cells that have been contacted with one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population express CD62L, CD45RA and/or CD45RO than a population of T cells that have been contact with one volume of anti-CD3/28 nanomatrix, e.g. T Cell Transact™ to 17 volumes or more (e.g. 17.5, 20, 30 or 40 volumes) of the T cell population.

### T Cell Therapy

The instant disclosure provides methods for increasing the percentage of immature, less differentiated T cells in a population by contacting the one or more T cells with a ratio of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell as compare to a population of T cells that have been contact with a ratio of one volume of anti-CD3/28 nanomatrix, e.g. T Cell Transact™ to 17 volumes or more (e.g. 17.5, 20, 30 or 40 volumes) of the T cell population. In some embodiments, the method further includes administering the one or more T cells prepared using the methods provided herein to a subject in need thereof. One of skill in the art will appreciate that the one or more T cells produced by the methods provided herein can be used in any method of treating a patient comprising administering to the patient one or more T cells.

For example, and without limitation, the methods described herein can enhance the effectiveness of a T cell therapy, which can be an adoptive T cell therapy selected from the group consisting of tumor-infiltrating lymphocyte (TIL) immunotherapy, autologous cell therapy, engineered autologous cell therapy (eACT™), allogeneic T cell transplantation, engineered allogeneic T cell therapy, non-T cell transplantation, and any combination thereof. Adoptive T cell therapy broadly includes any method of selecting, enriching in vitro, and administering to a patient autologous or allogeneic T cells that recognize and are capable of binding tumor cells. TIL immunotherapy is a type of adoptive T cell therapy, wherein lymphocytes capable of infiltrating tumor tissue are isolated, enriched in vitro, and administered to a patient. The TIL cells can be either autologous or allogeneic. Autologous cell therapy is an adoptive T cell therapy that involves isolating T cells capable of targeting tumor cells from a patient, enriching the T cells in vitro, and administering the T cells back to the same patient. Allogeneic T cell transplantation can include transplant of naturally occurring T cells expanded ex vivo or genetically engineered T cells. Non-T cell transplantation can include autologous or allogeneic therapies with non-T cells such as, but not limited to, natural killer (NK) cells.

In one particular embodiment, a T cell therapy of the present disclosure is engineered allogeneic T cell therapy. According to this embodiment, the method can include collecting blood cells from a donor. The isolated blood cells (e.g., T cells) can then be genetically engineered to reduce expression of one or more naturally-occurring proteins (e.g., endogenous TCR), contacted with of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population at a cell density of at least about 0.2×10⁶ cells/ml to about 5.8×10⁶ cells/ml. The T cells can be engineered to express a chimeric antigen receptor (“engineered CAR T cells”) or T cell receptor (“engineered TCR T cells”). In one particular embodiment, the engineered allogeneic CAR T cells or the engineered TCR T cells that were contacted of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population are subsequently administered to a subject. In some embodiments, the engineered T cells are directed to a solid or liquid tumor in the subject.

In one embodiment, the one or more T cells can be engineered to express a chimeric antigen receptor. The chimeric antigen receptor can comprise a binding molecule to a tumor antigen. The binding molecule can be an antibody or an antigen binding molecule thereof.

For example, the antigen binding molecule can be selected from scFv, Fab, Fab′, Fv, F(ab′)₂, camelid and dAb, and any fragments or combinations thereof.

The chimeric antigen receptor can be engineered to target a particular tumor antigen. In some embodiments, the tumor antigen is selected from BCMA, EGFRvIII, Flt-3, WT-1, CD20, CD23, CD30, CD38, CD70, CD33, CD133, MHC-WT1, TSPAN10, MHC-PRAME, Liv1, ADAM10, CHRNA2, LeY, NKG2D, CS1, CD44v6, ROR1, CD19, Claudin-18.2 (Claudin-18A2 or Claudin18 isoform 2), DLL3 (Delta-like protein 3, Drosophila Delta homolog 3, Delta3), Muc17 (Mucin17, Muc3, Muc3), FAP alpha (Fibroblast Activation Protein alpha), Ly6G6D (Lymphocyte antigen 6 complex locus protein G6d, c6orf23, G6D, MEGT1, NG25), RNF43 (E3 ubiquitin-protein ligase RNF43, RING finger protein 43), ErbB2 (HER2/neu), carcinoembryonic antigen (CEA), epithelial cell adhesion molecule (EpCAM), epidermal growth factor receptor (EGFR), CD40, disialoganglioside GD2, GD3, C-type lectin-like molecule-1 (CLL-1), ductal-epithelial mucine, gp36, TAG-72, glycosphingolipids, glioma-associated antigen, (3-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, survivin and telomerase, prostate-carcinoma tumor antigen-1 (PCTA-1), MAGE, ELF2M, neutrophil elastase, ephrin B2, CD22, insulin growth factor (IGF1)-1, IGF-II, IGFI receptor, mesothelin, a major histocompatibility complex (MHC) molecule presenting a tumor-specific peptide epitope, 5T4, O 1, Nkp30, tumor stromal antigens, the extra domain A (EDA) and extra domain B (EDB) of fibronectin and the AI domain of tenascin-C (TnC AI) and fibroblast associated protein (fap), LRP6, melanoma-associated Chondroitin Sulfate Proteoglycan (MCSP), MARTI, MUC1, LMP2, Idiotype, NY-ESO-1, Ras mutant, gp100, proteinase 3, bcr-ab1, tyrosinase, hTERT, EphA2, ML-TAP, ERG, NA17, PAX3, ALK, Androgen receptor, a lineage-specific or tissue specific antigen such as CD3, CD4, CD8, CD24, CD25, CD34, CD79, CD116, CD117, CD135, CD123, CD138, CTLA-4, B7-1 (CD80), B7-2 (CD86), endoglin, a major histocompatibility complex (MHC) molecule, MUC16, PSCA, Trop2, CD171 (L1CAM), CA9, STEAP1, VEGFR2, and any combination thereof.

The methods provided herein can involve a T cell therapy comprising the transfer of one or more T cells to a patient. The T cells can be administered at a therapeutically effective amount. For example, a therapeutically effective amount of T cells, e.g., engineered CAR⁺ T cells or engineered TCR⁺ T cells, can be at least about 10⁴ cells, at least about 10 cells, at least about 10⁶ cells, at least about 10⁷ cells, at least about 10⁸ cells, at least about 10⁹, or at least about 10¹⁰. In another embodiment, the therapeutically effective amount of the T cells, e.g., engineered CAR+ T cells or engineered TCR⁺ T cells, is about 10⁴ cells, about 10⁵ cells, about 10⁶ cells, about 10⁷ cells, or about 10⁸ cells. In one particular embodiment, the therapeutically effective amount of the T cells, e.g., engineered CAR⁺ T cells or engineered TCR⁺ T cells, is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

In some embodiments, the patient is preconditioned prior to administration of the T cell therapy. The patient can be preconditioned according to any methods known in the art, including, but not limited to, treatment with one or more chemotherapy drug and/or radiotherapy. In some embodiments, the preconditioning can include any treatment that reduces the number of endogenous lymphocytes, removes a cytokine sink, increases a serum level of one or more homeostatic cytokines or pro-inflammatory factors, enhances an effector function of T cells administered after the conditioning, enhances antigen presenting cell activation and/or availability, or any combination thereof prior to a T cell therapy.

### Cancer Treatment

The methods of the instant disclosure can be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In certain embodiments, the methods induce a complete response. In other embodiments, the methods induce a partial response.

One embodiment, the instant disclosure is directed to a method of treating a tumor in a subject in need of a T cell therapy comprising administering to the subject one or more T cells, wherein the one or more T cells have been contacted with of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population at a cell density of at least about 0.2×10⁶ cells/ml to about 5.8×10⁶ cells/ml. In another embodiment, the instant disclosure is directed to a method of reducing or decreasing the size of a tumor or inhibiting growth of a tumor in a subject in need of a T cell therapy comprising administering to the subject one or more T cells, wherein the one or more T cells have been contacted with of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population at a cell density of at least about 0.2×10⁶ cells/ml to about 5.8×10⁶ cells/ml.

Cancers that can be treated include tumors that are not vascularized, not yet substantially vascularized, or vascularized. The cancer can also include solid or non-solid tumors. In certain embodiments, the cancer can be selected from a tumor derived from acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies such as NHL, basal cell carcinoma, bile duct cancer, bladder cancer, bone cancer, osteosarcoma and malignant fibrous histiocytoma, brain stem glioma, brain tumors, breast cancer, bronchial tumors, Burkitt lymphoma, carcinoid tumors, central nervous system cancers, cervical cancer, chordoma, chronic lymphocytic leukemia (CLL), chronic myelogenous leukemia (CIVIL), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, Hodgkin's lymphoma, hypopharyngeal cancer, intraocular melanoma, islet cell tumors (endocrine pancreas), kaposi sarcoma, kidney cancer, Langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CIVIL), Myeloid leukemia, acute (AML), myeloma, multiple, myeloproliferative disorders, nasal cavity and paranasal sinus cancer, nasopharyngeal cancer, neuroblastoma, non-Hodgkin's lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sezary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenstrom macroglobulinemia, Wilms Tumor.

In one embodiment, the method can be used to treat a tumor, wherein the tumor is a lymphoma or a leukemia. Lymphoma and leukemia are cancers of the blood that specifically affect lymphocytes. All leukocytes in the blood originate from a single type of multipotent hematopoietic stem cell found in the bone marrow. This stem cell produces both myeloid progenitor cells and lymphoid progenitor cell, which then give rise to the various types of leukocytes found in the body. Leukocytes arising from the myeloid progenitor cells include T lymphocytes (T cells), B lymphocytes (B cells), natural killer cells, and plasma cells. Leukocytes arising from the lymphoid progenitor cells include megakaryocytes, mast cells, basophils, neutrophils, eosinophils, monocytes, and macrophages. Lymphomas and leukemias can affect one or more of these cell types in a patient.

Accordingly, in some embodiments, the method can be used to treat a lymphoma or a leukemia, wherein the lymphoma or leukemia is a B cell malignancy. Examples of B cell malignancies include, but are not limited to, Non-Hodgkin's Lymphomas (NHL), Small lymphocytic lymphoma (SLL/CLL), Mantle cell lymphoma (MCL), FL, Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma, and Lymphoblastic lymphoma. In some embodiments, the lymphoma or leukemia is selected from B-cell chronic lymphocytic leukemia/small cell lymphoma, B-cell prolymphocytic leukemia, lymphoplasmacytic lymphoma (e.g., Waldenstrom macroglobulinemia), splenic marginal zone lymphoma, hairy cell leukemia, plasma cell neoplasms (e.g., plasma cell myeloma (i.e., multiple myeloma), or plasmacytoma), extranodal marginal zone B cell lymphoma (e.g., MALT lymphoma), nodal marginal zone B cell lymphoma, follicular lymphoma (FL), transformed follicular lymphoma (TFL), primary cutaneous follicle center lymphoma, mantle cell lymphoma, diffuse large B cell lymphoma (DLBCL), Epstein-Barr virus-positive DLBCL, lymphomatoid granulomatosis, primary mediastinal (thymic) large B-cell lymphoma (PMBCL), Intravascular large B-cell lymphoma, ALK+ large B-cell lymphoma, plasmablastic lymphoma, primary effusion lymphoma, large B-cell lymphoma arising in HHV8-associated multicentric Castleman's disease, Burkitt lymphoma/leukemia, T-cell prolymphocytic leukemia, T-cell large granular lymphocyte leukemia, aggressive NK cell leukemia, adult T-cell leukemia/lymphoma, extranodal NK/T-cell lymphoma, enteropathy-associated T-cell lymphoma, Hepatosplenic T-cell lymphoma, blastic NK cell lymphoma, Mycosis fungoides/Sezary syndrome, Primary cutaneous anaplastic large cell lymphoma, lymphomatoid papulosis, Peripheral T-cell lymphoma, Angioimmunoblastic T cell lymphoma, Anaplastic large cell lymphoma, B-lymphoblastic leukemia/lymphoma, B-lymphoblastic leukemia/lymphoma with recurrent genetic abnormalities, T-lymphoblastic leukemia/lymphoma, and Hodgkin lymphoma. In some embodiments, the cancer is refractory to one or more prior treatments, and/or the cancer has relapsed after one or more prior treatments.

In certain embodiments, the cancer is selected from follicular lymphoma, transformed follicular lymphoma, diffuse large B cell lymphoma, and primary mediastinal (thymic) large B-cell lymphoma. In one particular embodiment, the cancer is diffuse large B cell lymphoma.

In some embodiments, the cancer is refractory to or the cancer has relapsed following one or more of chemotherapy, radiotherapy, immunotherapy (including a T cell therapy and/or treatment with an antibody or antibody-drug conjugate), an autologous stem cell transplant, or any combination thereof. In one particular embodiment, the cancer is refractory diffuse large B cell lymphoma.

In some embodiments, the cancer is treated by administering the one or more T cells to a subject, wherein the one or more T cells have been contacted with of one volume of anti-CD3/28 nanomatrix, e.g. T Cell Transact™ to 17 volumes or less (e.g. 1, 2, 3, 4, 5, 6, 7, 8, 9, 10, 12, 13, 14, 15 or 16 volumes) of the T cell population at a cell density of at least about 0.2×10⁶ cells/ml to about 5.8×10⁶ cells/ml. In some embodiments, the one or more T cells comprise engineered CAR cells or engineered TCR cell. In one embodiment, the engineered CAR cells or the engineered T cells treat a tumor in the subject.

The present invention is further illustrated by the following examples which should not be construed as further limiting. The contents of all references cited throughout this application are expressly incorporated herein by reference.

The following examples are intended to illustrate various embodiments of the instant disclosure. As such, the specific embodiments discussed are not to be construed as limitations on the scope of the instant disclosure. For example, although the Examples below are directed to T cells transduced with an anti-CD19 chimeric antigen receptor (CAR), one skilled in the art would understand that the methods described herein can apply to T cells transduced with any CAR. It will be apparent to one skilled in the art that various equivalents, changes, and modifications can be made without departing from the scope of instant disclosure, and it is understood that such equivalent embodiments are to be included herein. Further, all references cited in the disclosure are hereby incorporated by reference in their entirety, as if fully set forth herein.

### Embodiments

E1. A method for increasing a percentage of stem memory T cells in a cell population comprising 1) contacting a volume of an anti-CD3/CD28 nanomatrix with a volume of a starting cell population at a volumetric ratio wherein the starting cell population is at a cell density of at least about 0.2×10⁶ cells/ml to about 5.8×10⁶ cells/ml and the volumetric ratio is 1 volume of anti-CD3/CD28 nanomatrix to 17.4 volumes or less of the starting cell population, and 2) culturing the cell population in a culture medium, wherein the resulting T cell population comprises an increased percentage of stem memory T cells relative to a second T cell population wherein the second starting cell population of the same cell density is contacted with the anti-CD3/CD28 nanomatrix at a ratio of one volume of anti-CD3/CD28 nanomatrix to 17.5 or more volumes of the second starting cell population.

E2. The method of E1, wherein the nanomatrix is a Macs® GMP T Cell Transact™ nanomatrix.

E3. The method of any of the preceding embodiment, wherein the starting cell populations are obtained from peripheral blood, bone marrow, lymph node tissue, cord blood, thymus tissue, tissue from a site of infection, ascites, pleural effusion spleen tissue, a tumor, mesenchymal tissue, a T cell line, or an artificial thymic organoid (ATO) cell culture system.

E4. The method of any of the preceding embodiments, wherein the starting cell populations are selected from the group consisting of: T lymphocytes, B lymphocytes, helper T cells, tumor infiltrating lymphocytes, memory T cells, cytotoxic T cells, natural killer T cells, peripheral blood lymphocytes, tumor infiltrating leukocytes, peripheral blood mononuclear cells, dendritic cells, cord blood stem cells, pluripotent stem cells and mesenchymal stem cells.

E5. The method of any of the preceding embodiments, wherein the starting cell populations are peripheral blood mononuclear cells, positively selected CD3, CD4, or CD8 T cells or negatively selected CD3, CD4, or CD8 T cells.

E6. The method of any of the preceding embodiments, wherein the starting cell populations are peripheral blood mononuclear cells.

E7. The method of any of embodiments 1 to 4 wherein the starting cell populations are purified T cell populations.

E8. The method of any of the preceding embodiments, wherein the starting cell density is at least about 0.50×10⁶ to about 6.00×10⁶, about 0.56×10⁶ to about 5.72×10⁶, about 0.80×10⁶ to about 4.0×10⁶, about 1.00×10⁶ to about 3.0×10⁶, about 2.00×10⁶ to about 3. 5×10⁶, or about 2.50×10⁶ to about 3.5×10⁶.

E9. The method of any of the preceding embodiments, wherein the cell density is about 2.86×10⁶ cells/ml.

E10. The method of any of embodiments 1 to 9, wherein the starting cell density is about 3.00×10⁶ cells/ml.

E11. The method of any of embodiments 1 to 9, wherein the starting cell density is at least about 0.28×10⁶ to about 2.86×10⁶, 0.25×10⁶ to about 2.00×10⁶, about 0.5×10⁶ to about 2×10⁶, about 1.00×10⁶ to about 2.00×10⁶, about 0.80×10⁶ to about 1.5×10⁶, or about 1.20×10⁶ to about 1.5×10⁶ cells/ml.

E12. The method of any of embodiments 1 to 8 or 11, wherein the cell density is about 1.43×10⁶ cells/ml.

E13. The method of any of embodiments 1 to 8 or 11, wherein the starting cell density is about 1.00×10⁶ cells/ml.

E14. The method of any of the preceding embodiments wherein the volumetric ratio is about 1:5, 1:6, 1:7, 1:8, 1:9, 1:10, 1:11, 1.12, 1:13, 1:14, 1:15, 1:16 or 1:17 of anti-CD3/28 nanomatrix volume to starting cell population volume.

E15. The method of any of the preceding embodiments, further comprising transducing the starting cell populations before, during or after culturing the starting cell populations with the anti-CD3/CD28 nanomatrix.

E16. The method of any of embodiment 15 wherein the starting cell populations are transduced with a lentivirus vector, retrovirus vector and/or adenovirus associated vector.

E17. The method of any of the preceding embodiments, further comprising transfecting the starting cell populations before, during or after culturing the starting cell populations with the anti-CD3/CD28 nanomatrix.

E18. The method of embodiment 17, wherein the transfecting step comprises TALEN® mRNA electroporation (EP) and/or CRISPER Cas9 electroporation.

E19. The method of embodiment 18, wherein the transfecting step comprises disrupting the TCRαβ gene and/or β2M gene.

E20. The method of any of the preceding embodiments wherein the starting cell populations are cultured for about 8, 9, 10, 11, 12 ,13 ,14, 15, 16, 17, 18, 19 or 20 days.

E21. The method of any one of any of the preceding embodiments, wherein the resulting T cell population express one or more markers indicative of undifferentiated or immature T cells.

E22. The method of embodiment 21, wherein the one or more markers indicative of undifferentiated or immature T cells are selected from the group consisting of CD62L, CD45RA, CD45RO, and any combination thereof.

E23. The method of any of the previous embodiments wherein the resulting T cell population comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% more stem memory T cells than the second resulting T cell population.

E24. The method of any of the preceding embodiments wherein the starting cell population volumetric ratio is 1:5 and the second starting cell population volumetric ratio is 1:20, 1:25 or 1:50.

E25. The method of any of embodiments 1 to 24, wherein the starting cell population volumetric ratio is 1:10 and the second starting cell population volumetric ratio is 1:20, 1:25 or 1:50.

E26. The method of any of embodiments 1 to 24, wherein the starting cell population volumetric ratio is 1:15 and the second starting cell population volumetric ration is 1:20, 1:25 or 1:50.

E27. The method of any of embodiments 16 to 26, wherein the lentivirus comprises a heterologous gene encoding a cell surface receptor.

E28. The method of embodiment 27, wherein the cell surface receptor is capable of binding an antigen on the surface of a target cell.

E29. The method of embodiment 28, wherein the target cell is a tumor cell.

E30. The method of 28 or 29, wherein the cell surface receptor is a T cell receptor (TCR) or a chimeric antigen receptor (CAR).

E31. The method of embodiment 30, wherein the TCR or the CAR is capable of binding an antigen selected from the group consisting of 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-ab1 (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2, -3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100 kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), .kappa.-light chain, LAGE (L antigen), LDLR/FUT (low density lipid receptor/GDP-L-fucose: b-D-galactosidase 2-a-Lfucosyltransferase), LeY (Lewis-Y antibody), L1CAM (L1 cell adhesion molecule), MAGE (melanoma antigen), MAGE-A1 (Melanoma-associated antigen 1), MAGE-A3, MAGE-A6, mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1, -2, -3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-ab1 (protein of 190 KD bcr-ab1), Pm1/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), SSX1, -2, -3, 4 (synovial sarcoma X1, -2, -3, -4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), WT1 (Wilms' tumor gene), CD70, FLT3, DLL3 and any combination thereof.

E33. The method of any of the preceding embodiments, further comprising the administering a therapeutically effective amount of the resulting T cells to a subject in need thereof.

E34. A method of treating a tumor in a subject in need of T cell therapy comprising administering to the subject one or more of the resulting T cells of any of embodiments preceding embodiments.

E35. A method of reducing or decreasing the size of a tumor or inhibiting the growth of at tumor in a subject in need of T cell therapy comprising administering to the subject one or more of the resulting T cells of any of the preceding embodiments.

E36. The method of any of embodiments 29 to 35, wherein the tumor is a cancer selected from bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, multiple myeloma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B cell malignancies, and any combination thereof.

E37. A method for increasing a percentage of stem memory T cells in a PBMC population comprising, 1) contacting a volume of an anti-CD3/CD28 nanomatrix with a volume of a starting PBMC population at a volumetric ratio wherein the starting PBMC population is at a cell density of at least about 0.5×10⁶ cells/ml to about 2.0×10⁶ cells/ml and the volumetric ratio is 1 volume of anti-CD3/CD28 nanomatrix to 5 or 10 volumes of the starting cell population, and 2) culturing the PBMC population in a culture medium for 14 to 18 days, wherein the resulting PBMC population comprises an increased percentage of stem memory T cells relative to a second PBMC population wherein the second starting PBMC population of the same cell density is contacted with the anti-CD3/CD28 nanomatrix at a ratio of one volume of anti-CD3/CD28 nanomatrix to 20 volumes of the second starting PBMC population and cultured for the same number of days.

E38. A method for increasing a percentage of stem memory T cells in a purified T cell population comprising, 1) contacting a volume of an anti-CD3/CD28 nanomatrix with a volume of a starting purified T cell population at a volumetric ratio wherein he starting T cell population is at a cell density of at least about 0.80×10⁶ cells/ml to about 1.60×10⁶ cells/ml and the volumetric ratio is 1 volume of anti-CD3/CD28 nanomatrix to 10 or 15 volumes of the starting cell population, and 2) culturing the PBMC population in a culture medium for 11 to 18 days, wherein the resulting purified T cell population comprises an increased percentage of stem memory T cells relative to a second purified T cell population wherein the second starting purified T cell population of the same cell density is contacted with the anti-CD3/CD28 nanomatrix at a ratio of one volume of anti-CD3/CD28 nanomatrix to 20, 25 or 50 volumes of the second starting T cell population and cultured for the same number of days.

E39. A method for increasing a total number of T cells in a T cell population comprising, 1) contacting a volume of an anti-CD3/CD28 nanomatrix with a volume of a starting T cell population at a volumetric ratio wherein the starting T cell population is at a cell density of at least about 0.80×10⁶ cells/ml to about 1.60×10⁶ cells/ml and the volumetric ratio is 1 volume of anti-CD3/CD28 nanomatrix to 10 or 15 volumes of the starting cell population, and 2) culturing the T cell population in a culture medium for 11 to 18 days, wherein the resulting T cell population comprises an increased number of T cells relative to a second T cell population wherein the second starting T cell population of the same cell density is contacted with the anti-CD3/CD28 nanomatrix at a ratio of one volume of anti-CD3/CD28 nanomatrix to 20, 25 or 50 volumes of the second starting T cell population and cultured for the same number of days.

### EXAMPLES

### Example 1

### Preparation of Ex Vivo Activated Allogeneic T Cells from a PBMC Population

Donor blood was collected and separated into its component parts by apheresis. PBMCs were then enriched over a Ficoll®-hypaque step gradient and cryopreserved. On Day 0, the Ficoll®-isolated frozen human PBMCs were thawed and washed one time with culture medium comprising 10% human serum. The cells were then cultured in medium with 5% human serum and incubated at 37° C. and 5% CO₂ overnight. On Day 1, the resulting PBMCs are at a cell density of about 3×10⁶ cells/ml or less and were divided into test fractions and activated as follows at 1.5×10⁶ cells/ml:

TABLE 1

| | | Activation |
|---|---|---|
| Test | | Culture |
| Group | Activation Conditions | Medium |
| 1 | mTransAct, CD3 1:100, CD28 1:200 | dilution |
| 2 | mTransact, CD3 1:100, CD28 1:200 | centrifugation |
| 3 | 1:5 volume hTransAct to cell culture volume | centrifugation |
| 4 | 1:10 volume hTransAct to cell culture volume | centrifugation |
| 5 | 1:10 volume hTransAct to cell culture volume | dilution |
| 6 | 1:20 volume hTransAct to cell culture volume | centrifugation |
| 7 | 1:40 volume hTransAct to cell culture volume | centrifugation |

For controls, test groups 1 to 2, the cells were cultured in the presence of mTransAct, which is provided in a mTransAct CD3/CD28 kit (catalog # 130-020-008, Miltenyi Biotec Inc., Auburn, Calif.) in which the anti-CD3 and anti-CD28 antibodies are mouse monoclonal antibodies against CD3 and CD28 respectively.

For test groups 3 through 5, the cells were cultured in the presence of Transact™ (Miltenyi Biotec Inc., Auburn, Calif.) in which the nanomatrix is conjugated to humanized anti-CD3 and anti-CD28 antibodies (hereinafter “h TransAct™”) at the indicated volumetric ratios.

For all test groups the cells were activated by the Transact™ to cell culture volumetric ratios as indicated in Table 1. The culture medium in which the cells were activated is composed of X-vivo 15 (Lonza), and 5% human serum (Gemini) plus IL-2 at 100 IU/ml (Miltenyi Biotec Inc).

On Day 4, the activation culture medium was either diluted with fresh culture medium or completely removed by centrifugation as indicated in Table 1. For test groups 1 and 5, the cells were diluted with at least one volume of fresh culture medium to one volumes of activation culture medium. For the remaining test groups, centrifugation was carried out at 540 g, for 5 minutes at room temperature. The cells were then washed one time with culture medium.

Further on Day 4, the activated cells can be genetically modified by lentiviral vector transduction for introduction of CAR by methods known in the art. Briefly, in test groups 1 and 5, at the dilution with fresh cell culture medium of the activation culture medium, a lentiviral vector (LVV #1831P, Lentigen Technology, Inc., Gaithersburg, Md.)) was added to the culture medium and the cells are transduced according to the manufacturer's instructions. For the remaining test groups the cells were transduced according to the manufacturer's instructions. Briefly, the cells were harvested, washed once, resuspended in culture medium to which a lentiviral vector (Lentigen Technology, Inc., Gaithersburg, Md.)) is added at 1-10% v/v of lentiviral vector to cell culture. In both methods the cell density of transduction was 1×10⁶ cells/mL.

The cells were expanded in T flasks from Day 4 to Day 6. On Day 6, the cells were further gene edited by TALEN® mRNA electroporation (EP) to disrupt the TCRαβ gene and knock out its gene expression. TALEN® mRNA electroporation were performed using AgilePulse® electroporation system (available from BTX®, a Division of Harvard Bioscience, Inc. Holliston, Mass.) according to the manufacturer's instructions.

The allogeneic T cells, including genetically modified allogeneic CAR-T cells, were expanded in G-Rex® 10 (Wilson Wolf Corporation, Saint Paul, Minn.) during Day 8 to Day 18 according to the manufacturer's instructions.

### Cell Growth and Yield

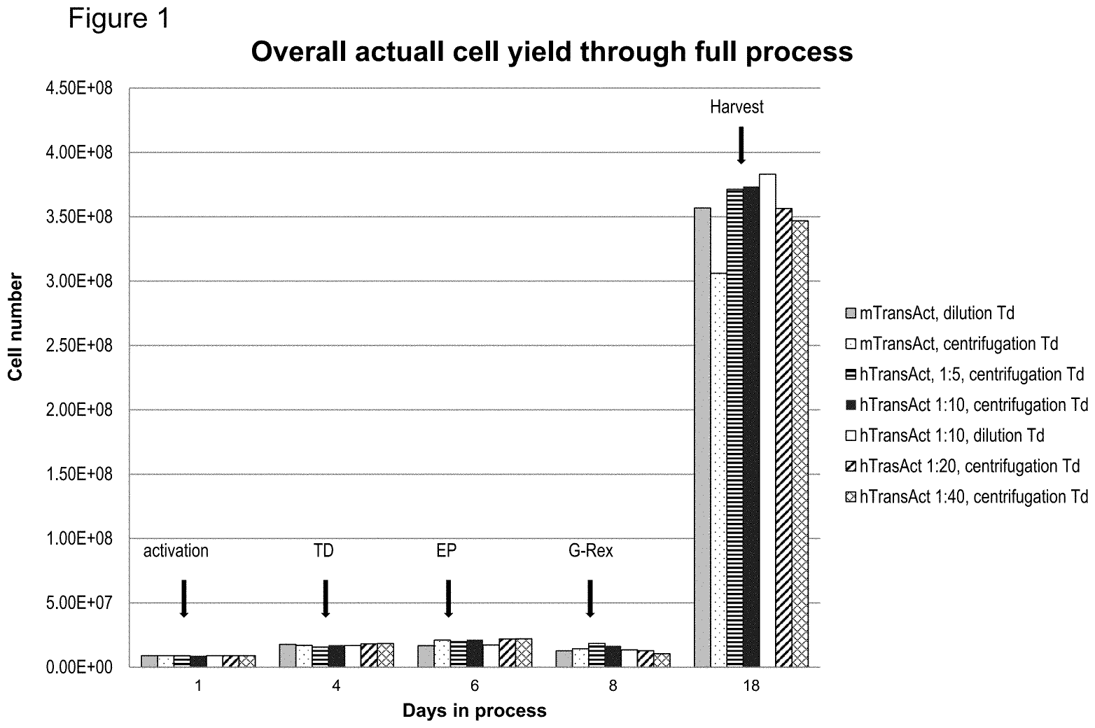

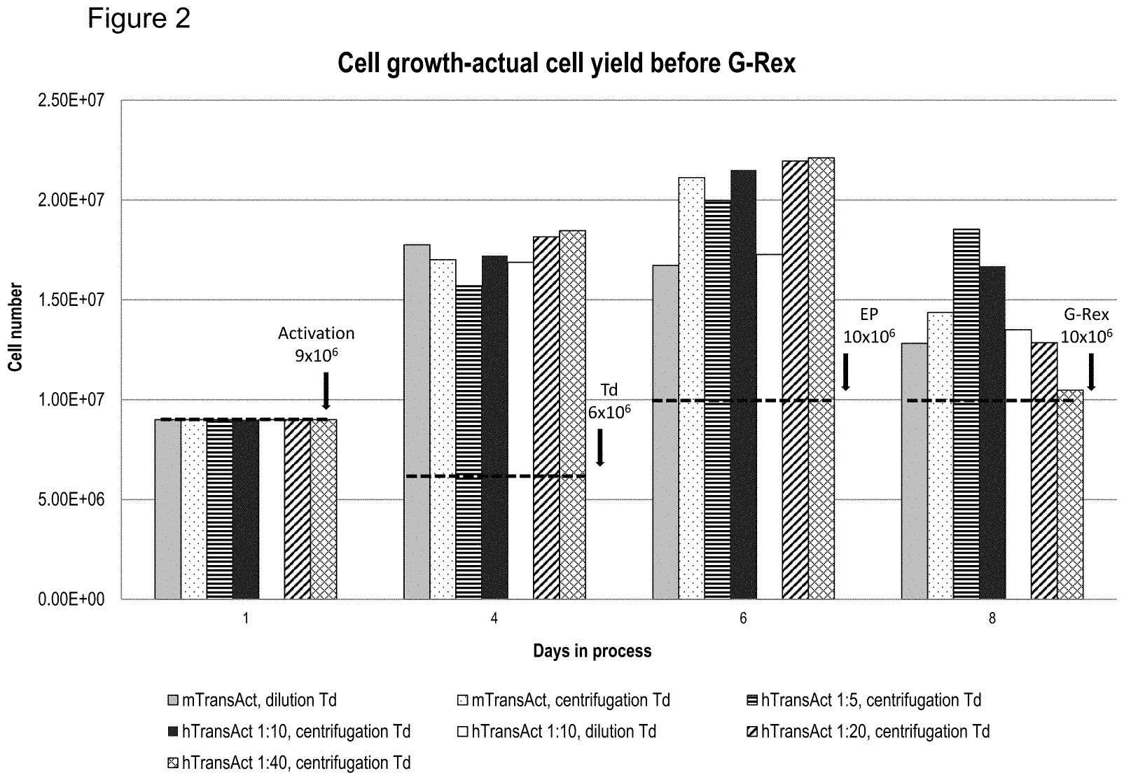

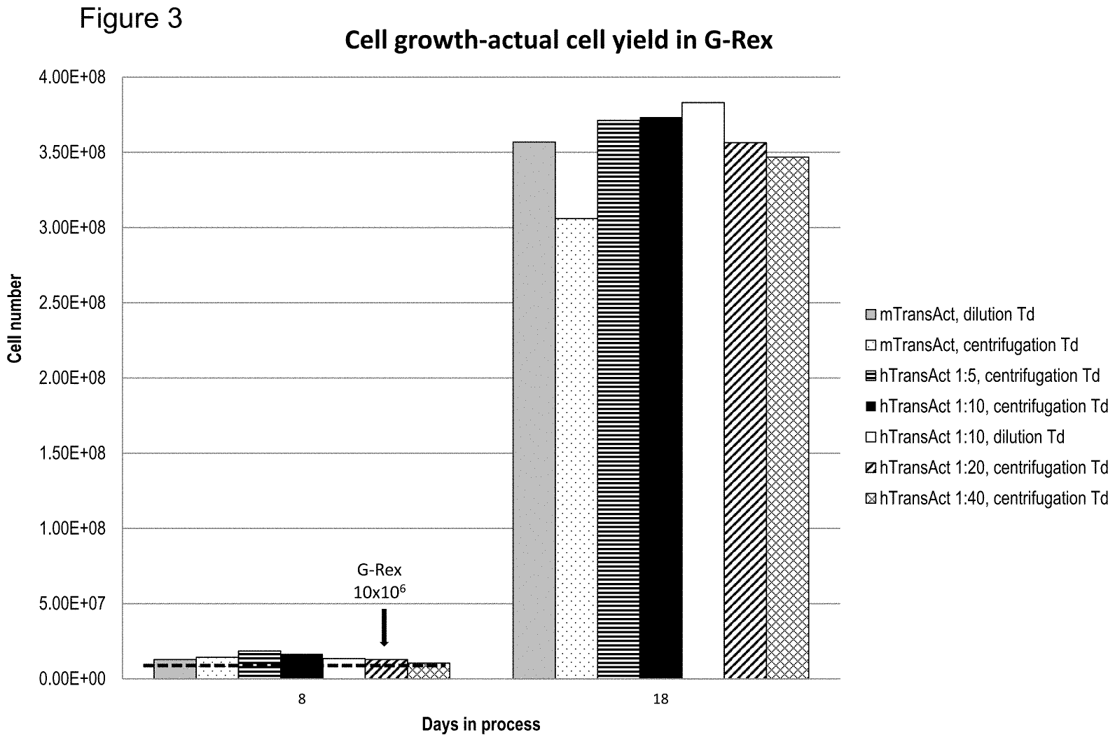

Actual cell yield was measured on Day 1, 4, 6, 8 and 18 using an automated cell counter NucleoCounter NC-200 (Chemometec, Allerod, Denmark). The results for Day 1 through Day 18 are illustrated in FIG. 1. The results for Days 1, 4, 6 and 8 before cell expansion in G-Rex® are illustrated in FIG. 2 while the results for expansion in G-Rex® from Day 8 through Day 18 are illustrated in FIG. 3.

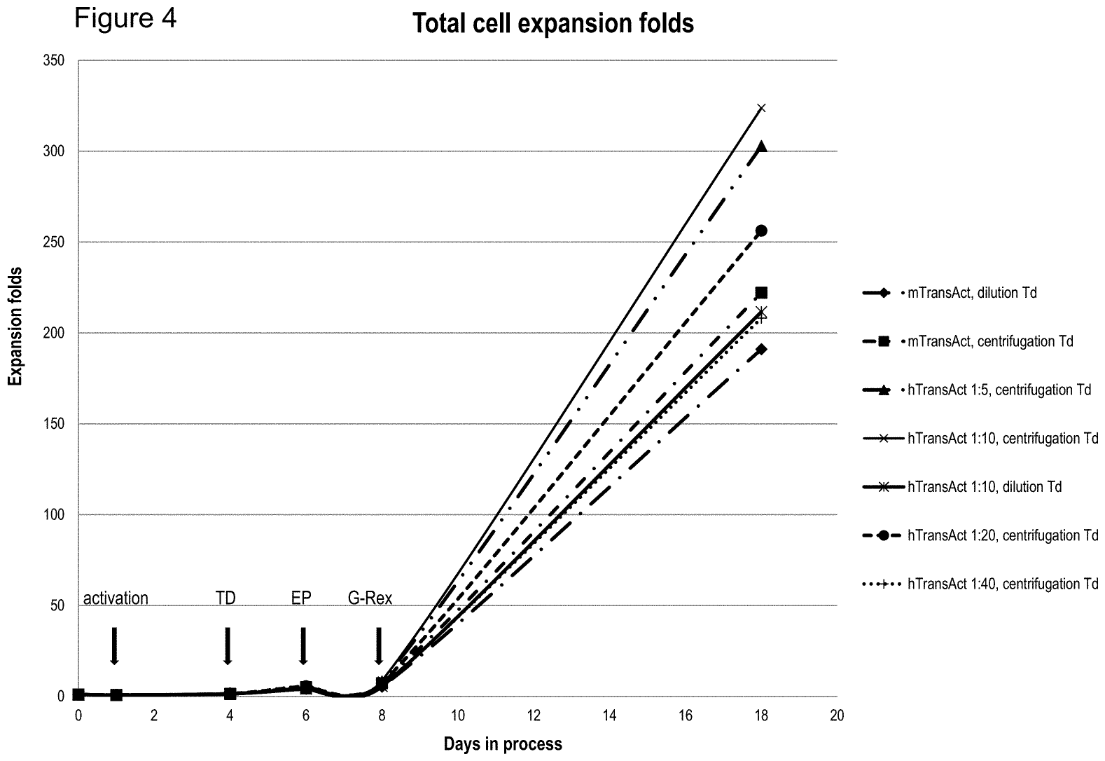

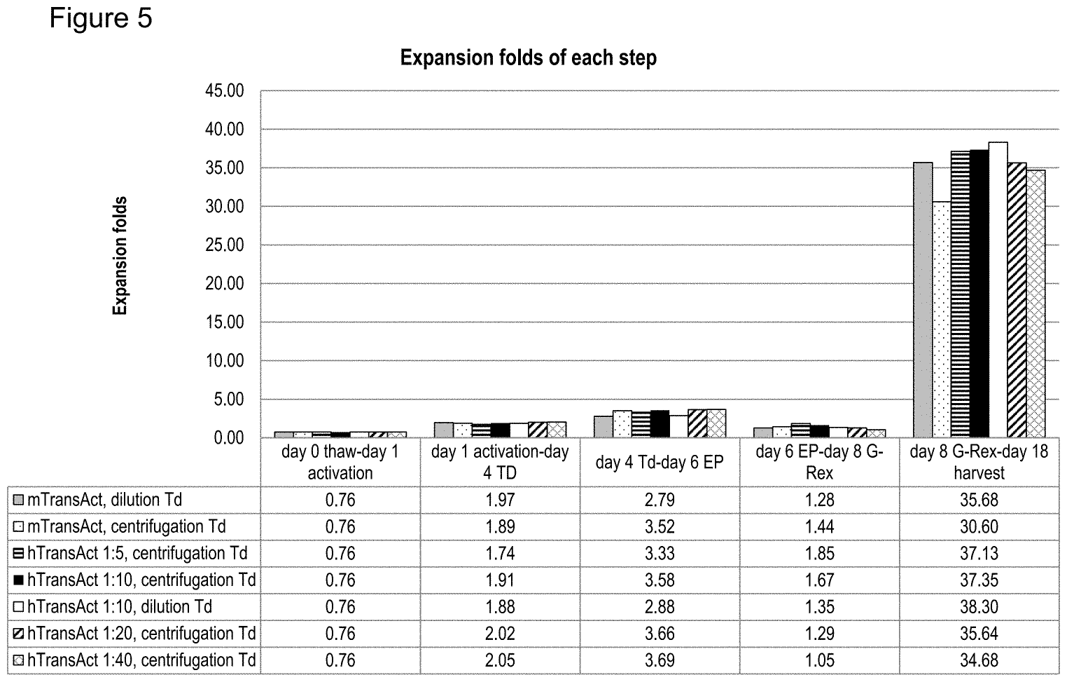

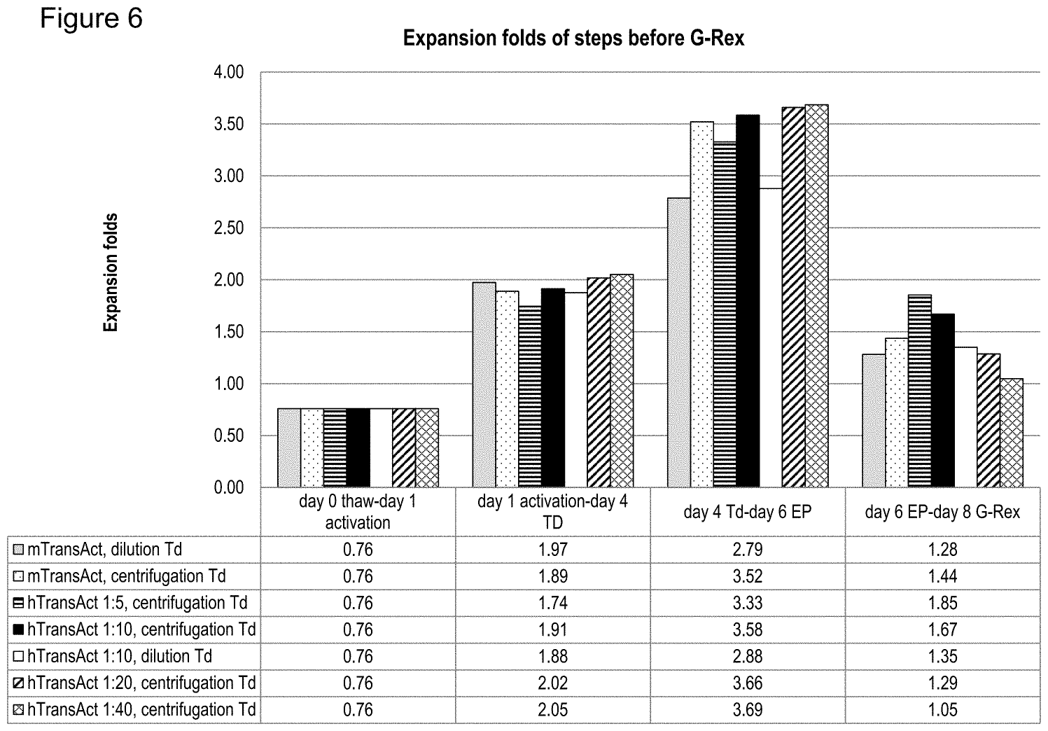

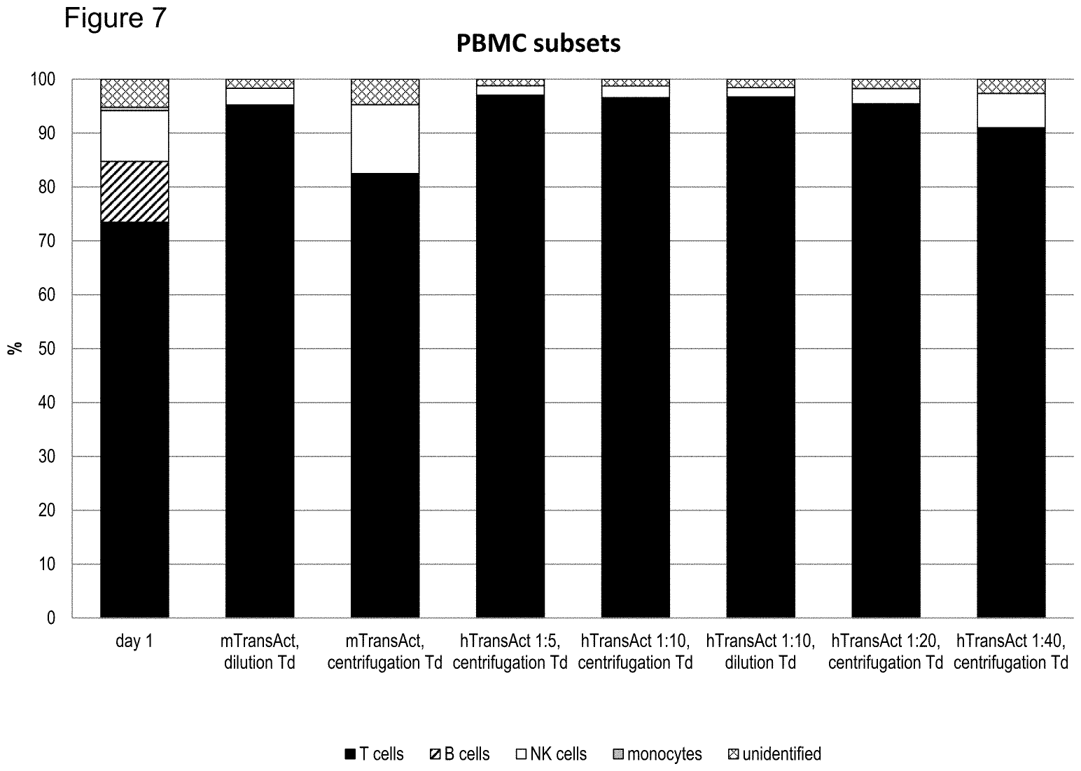

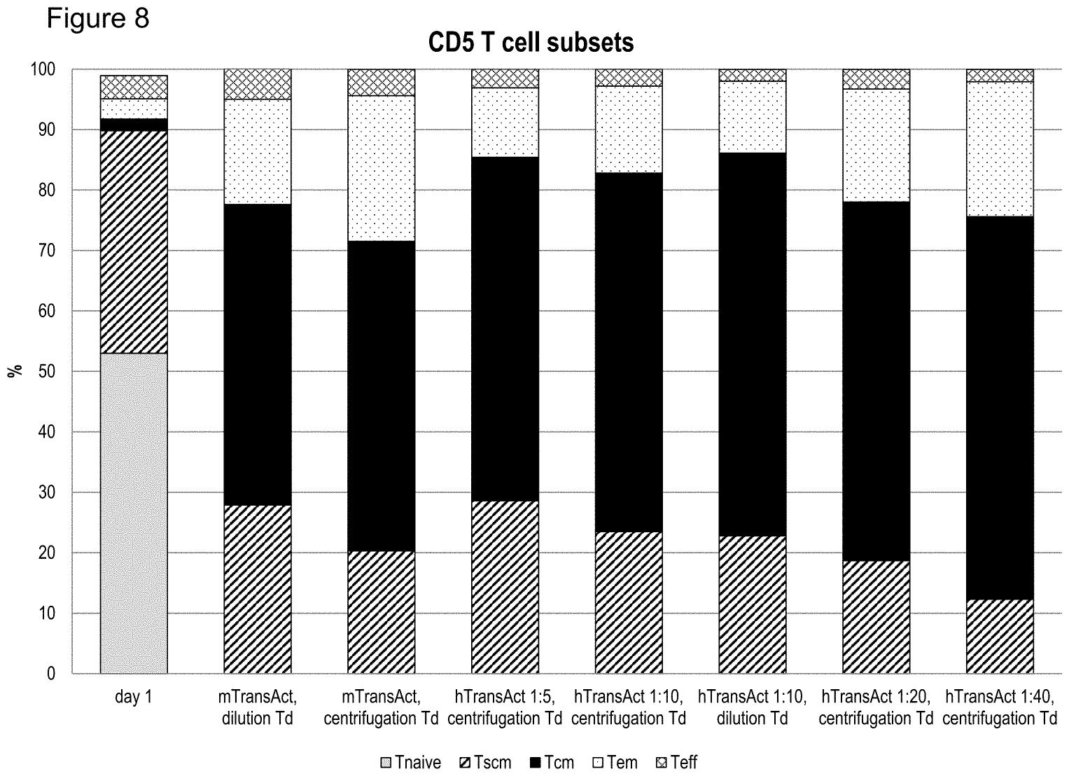

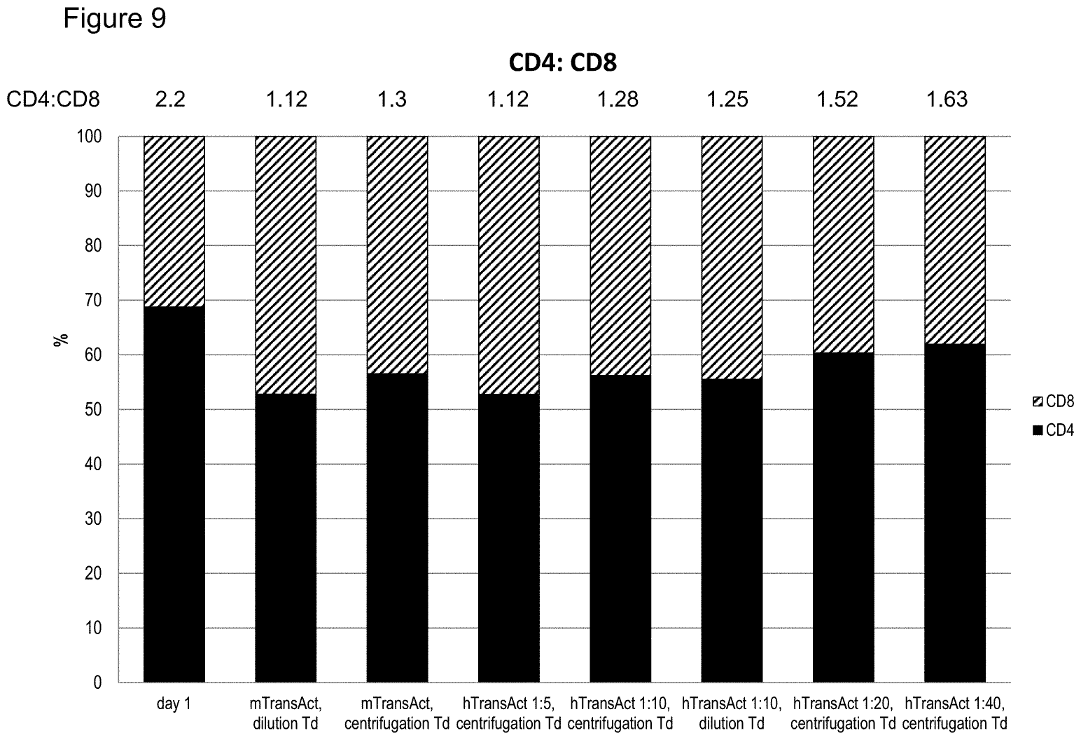

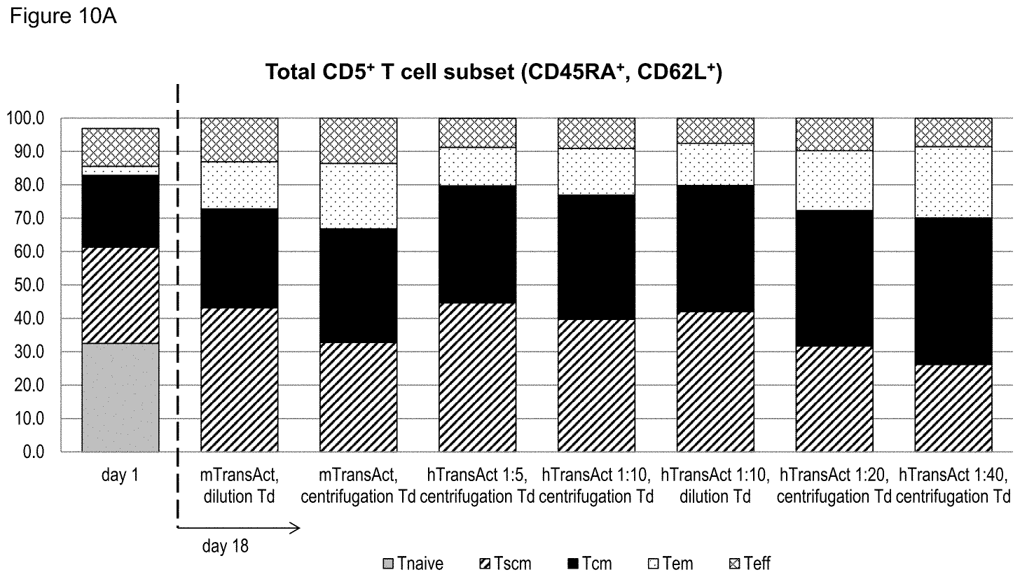

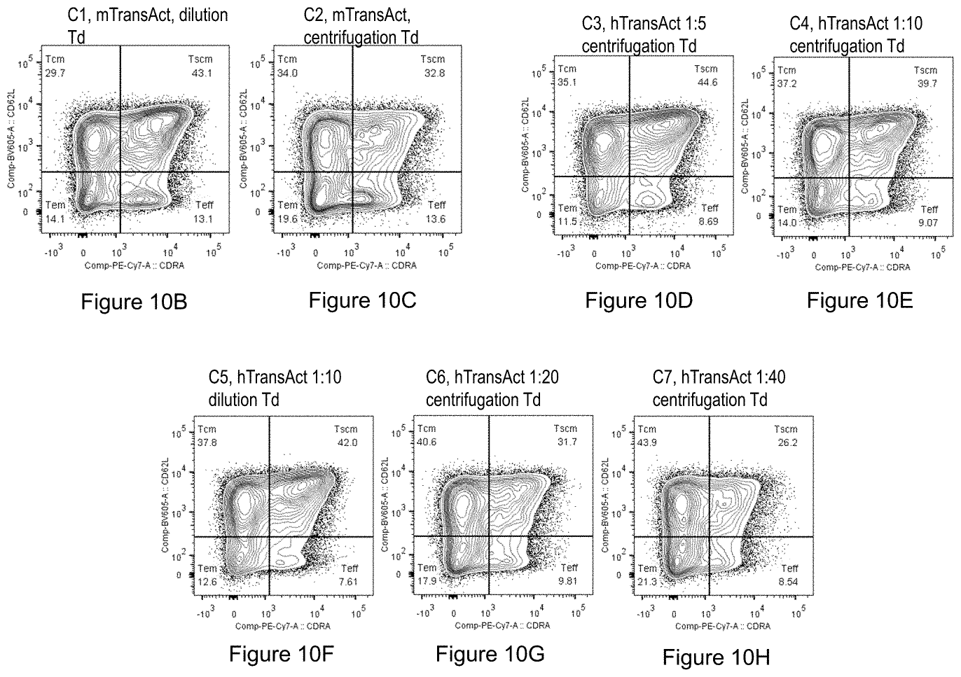

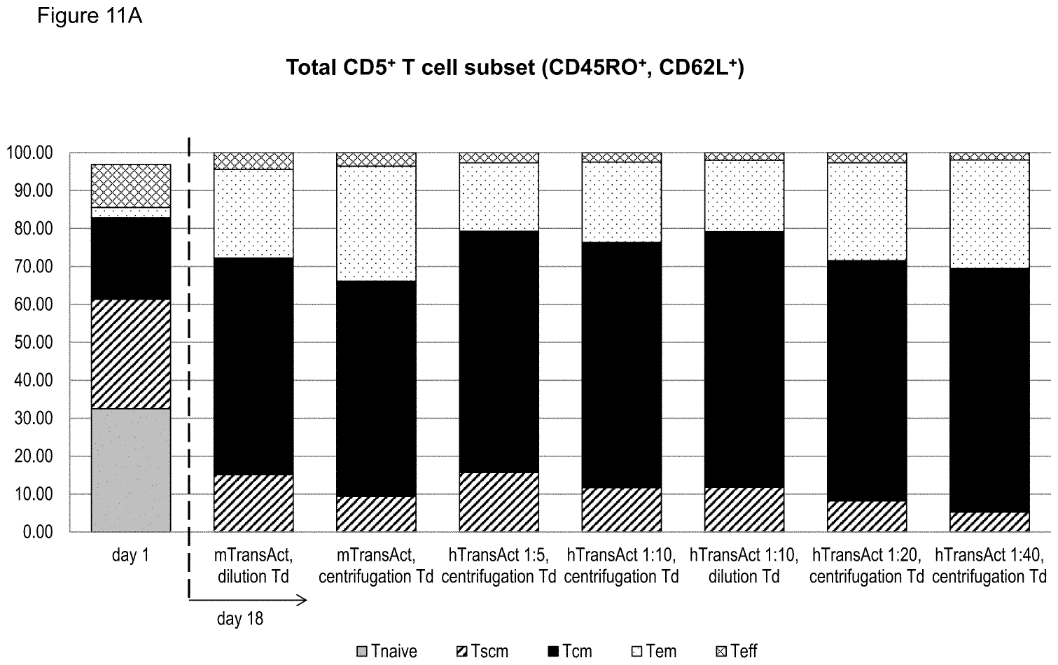

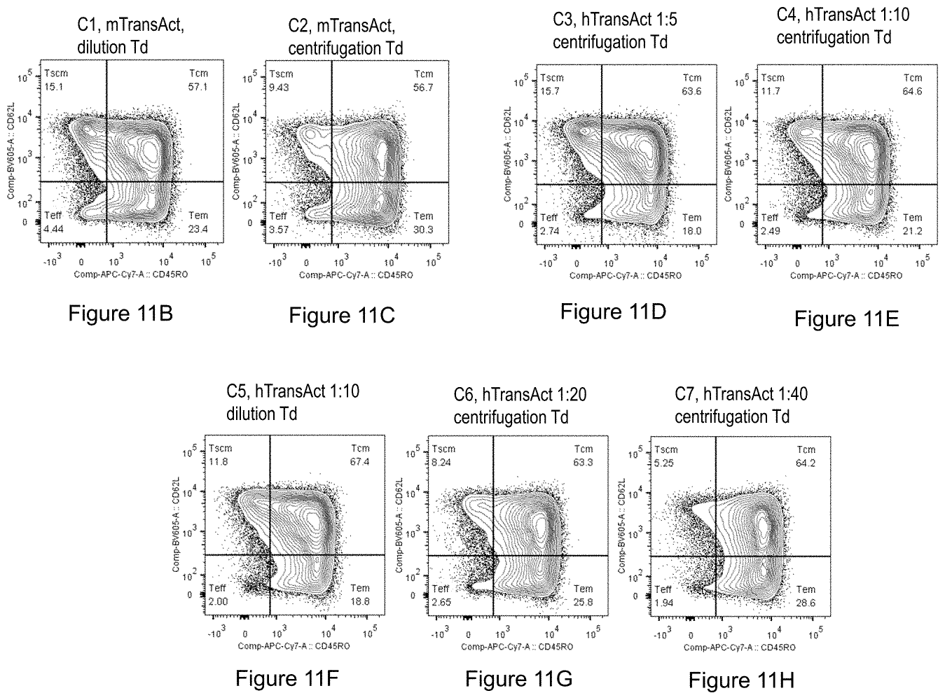

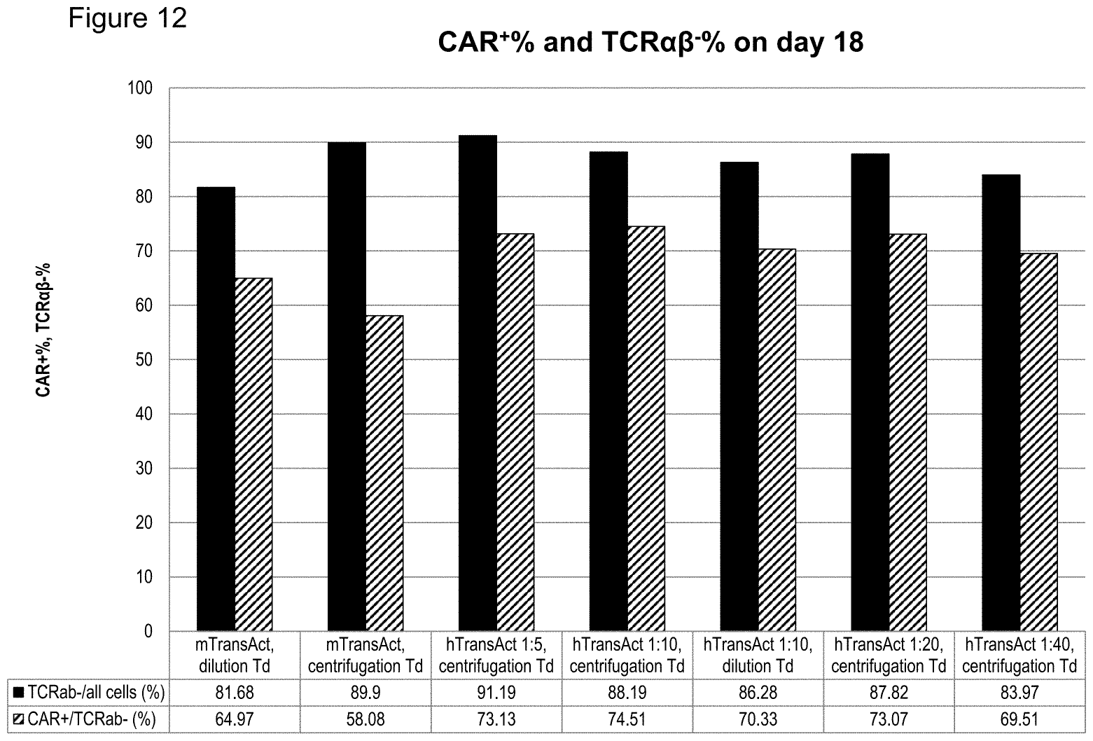

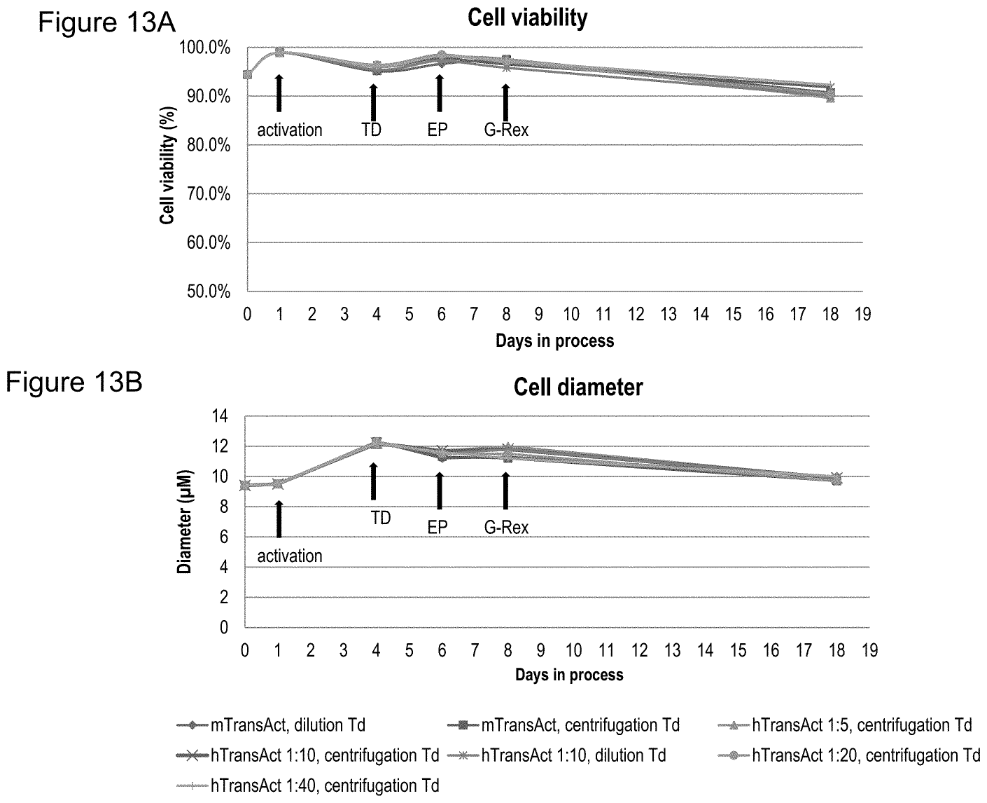

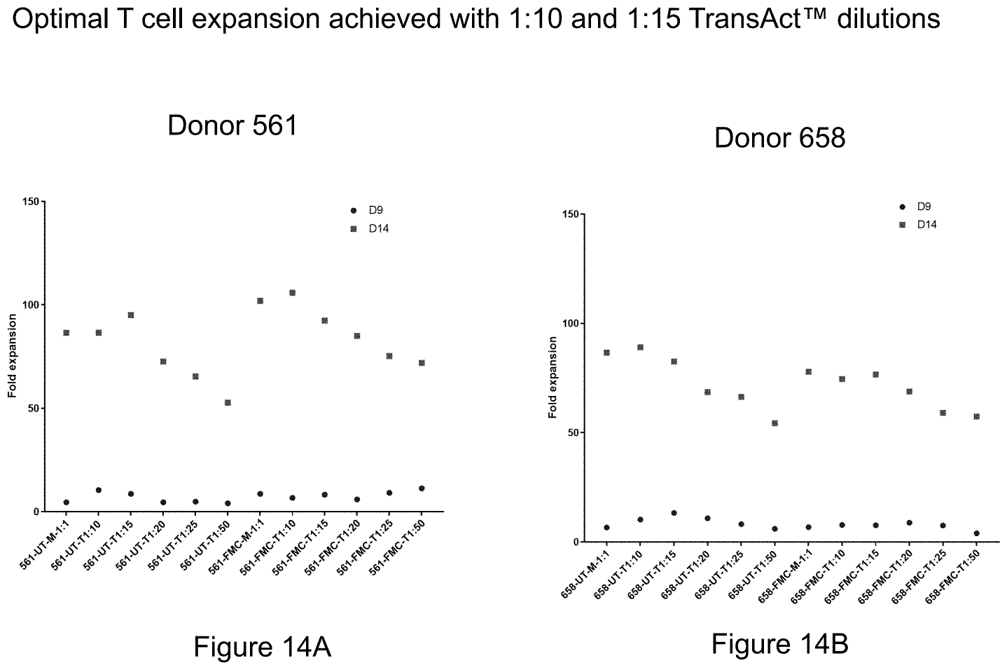

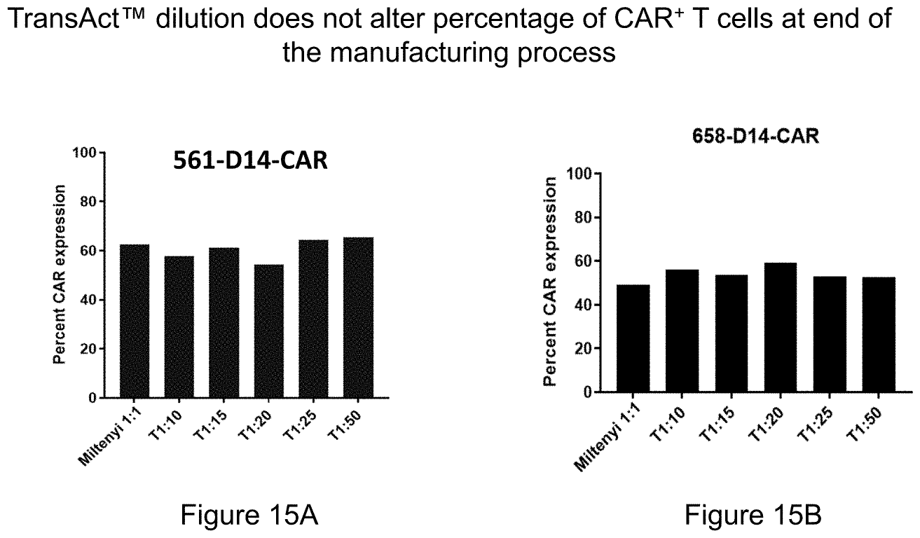

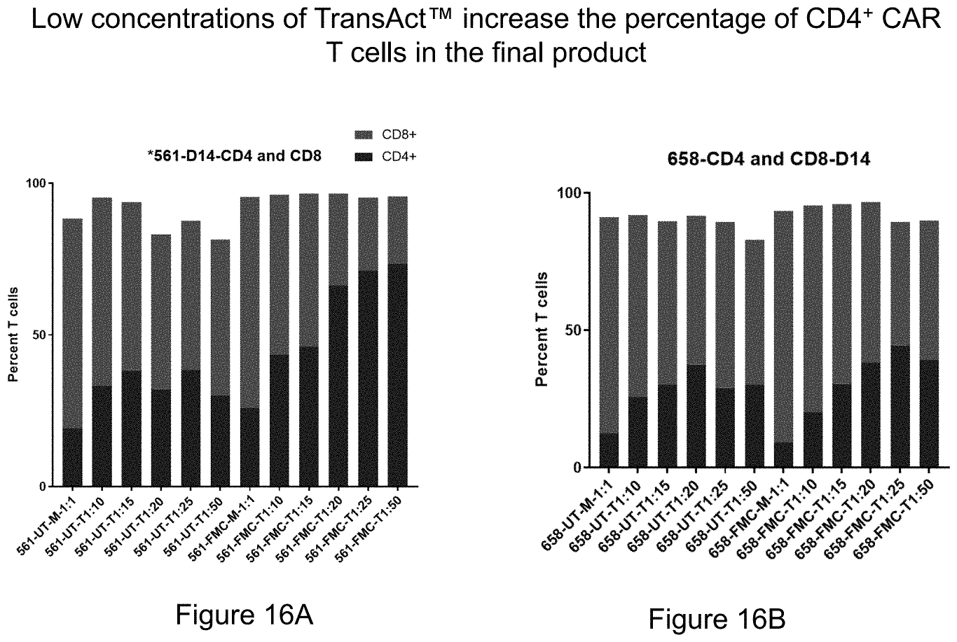

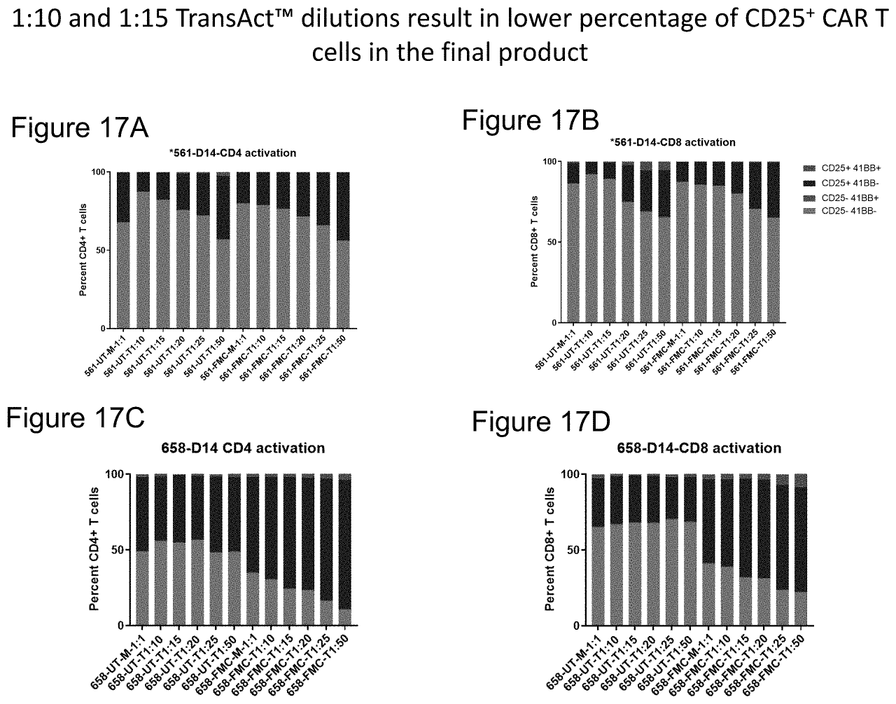

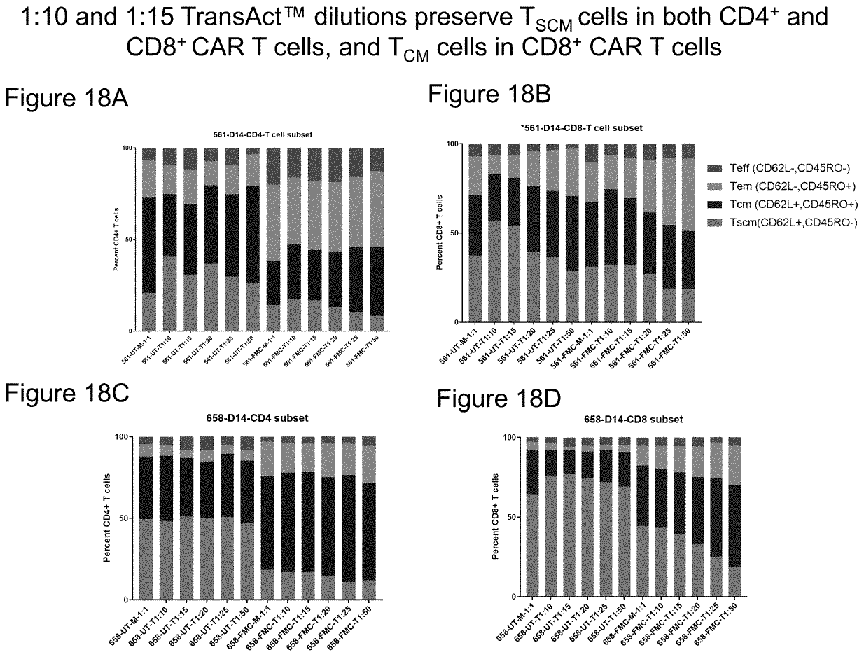

Total cell expansion folds were determined, and the results are illustrated in FIG. 4 for Day 1 through Day 18. FIG. 5 illustrates the expansion folds at each process step for each test group where test groups 3 to 5 have higher expansion folds than test groups 6 and 7 from Day 8 to 18. FIG. 6 illustrates the expansion fold results at each process step for each test group from Day 0 to Day 6.

### Cell Phenotype

The cell phenotypes are analyzed on Day 1 and Day 18. Cells are labeled with fluorescent antibodies against the relevant targets according to the manufacturer's instructions.

The fluorescent labeled cells were then analyzed by a LSRFortessa™ cytometer (BD Biosciences, Franklin Lakes, N.J.) according to the manufacturer's instructions and data were analyzed using FlowJo software Version 10 (FlowJo, LLC., Ashland, Oreg.).

The PBMC cell subsets were determined for each of the test groups and relevant controls on Day 1 using commercial antibodies against CD5 (BD Horizon), CD14 (Biolegend), CD56 (BD Biosciences), and CD19 (Biolegend).

The CD8 T cell subsets are determined for each of the test groups and relevant controls on Day 1 using commercial antibodies against CD8 (BD Biosciences), CD45RA (Biolegend), and CD62L (BD Biosciences)

The CD4:CD8 cell ratios were determined for each of the test groups and relevant controls on Day 1 using commercial antibodies against CD4 (BD Biosciences), and CD8 (BD Biosciences).

The total CD5⁺ cell subset which are CD45RA⁺, CD62L⁺, were determined for each of the test groups and relevant controls on Day 18 using commercial antibodies against CD5 (BD Horizon), CD45RA (Biolegend), and CD62L (BD Biosciences).

The total CD5⁺ cell subset which are CD45RO⁺, CD62L⁺, were determined for each of the test groups and relevant controls on Day 18 using commercial antibodies against CD5 (BD Horizon), CD45RO (Biolegend), and CD62L (BD Biosciences).

The percentage of CAR+ % and TCRαβ− % cells for each test group and relevant controls are determined on day 18 using a CAR anti-idiotype antibody conjugated to a fluorochrome and a commercial antibody against TCRαβ (Biolegend).

Cell viability was monitored from Day 0 to 18 for each test group and relevant controls using NucleoCounter® NC-200™ (Chemometec, Allerod, Denmark) according to the manufacturer's instructions.

Cell diameter was monitored from Day 0 to 18 for each test group and relevant controls using NucleoCounter® NC-2™ (Chemometec, Allerod, Denmark) according to the manufacturer's instructions.

### Example 2

### Preparation of Ex Vivo Activated Allogeneic T Cells from a Purified T Cell Population

Human peripheral blood mononuclear cells (PBMC) were isolated from buffy coats from anonymous blood donors using Ficoll®-Paque PLUS (GE Healthcare Life Sciences) and SepMate™-50 tubes (STEMCELL Technologies) following the protocol supplied by STEMCELL Technologies. Pan T cells were then isolated from the freshly prepared PBMC using the Pan T Cell Isolation Kit (Miltenyi Biotec) following the protocol supplied by Miltenyi Biotec. The pan T cells were stored in liquid nitrogen until ready for use.

Primary human pan T cells (10-30×10⁶) were thawed on Day 0, resuspended at approximately 2×10⁶ cells/mL in T cell transduction medium—X-Vivo™ 15 (Lonza) plus 10% HyClone fetal bovine serum (GE Healthcare Life Sciences)—and incubated for 30 min in a humidified incubator at 37° C. with 5% CO₂. After the 30 min incubation, the T cells were counted, and the cell density was adjusted using T cell transduction medium. The T cells were then mixed with either beads from the T Cell Activation/Expansion Kit (Miltenyi Biotec) at a 1:1 bead to T cell ratio or T Cell TransAct™ polymeric nanomatrix (Miltenyi Biotec) at the following volumetric dilutions: 1:10, 1:15, 1:20, 1:25, 1:50. Recombinant human IL-2 (Miltenyi Biotec) was added to a final concentration of 100 U/mL. The T cells were then returned to the 37° C. incubator.

Two days later (Day 2), the T cells were counted, resuspended at 5×10⁵ cells/mL in T cell transduction medium and fresh IL-2 was added. The T cells were then transduced with a lentiviral vector encoding the FMC63-41BB-CD3ζ anti-CD19 CAR and returned to the 37° C. incubator. Non-transduced (UT) controls were generated in parallel. On Day 5, the transduction efficiency was confirmed by flow cytometry and the FMC63-41BB-CD3ζ anti-CD19 CART cells, and UT control T cells, were transferred to a G-Rex® 6-well plate (Wilson Wolf). T cell culture medium—X-Vivo™ 15 (Lonza) plus 5% Human Serum AB (Off the Clot) (Gemini BioProducts) and 100 IU/mL recombinant human IL-2 (Miltenyi Biotec)—was added up to 35 mL.

Immunophenotyping was performed by flow cytometry using a LSRFortessa™ X-20 Cell Analyzer (BD Biosciences) and data were analyzed using FlowJo® v10 (FlowJo, LLC) software. The following antibodies were used: Alexa Fluor (AF) 700 anti-human CD25 antibody (BioLegend #302622); BUV395 anti-human CD62L antibody (BD Bioscience #565219); BV605 anti-human CD8a antibody (BioLegend #301040); BV786 anti-human CD4 antibody (BD Bioscience #563914); PE anti-human CD137 (4-1BB) antibody (BioLegend #309804); Peridinin chlorophyll (PerCP)/Cy5.5 anti-human CD45RO antibody (BioLegend #304222).

1. A method for increasing a percentage of stem memory T cells in a cell population comprising:
a) contacting a volume of an anti-CD3/CD28 nanomatrix with a volume of a starting cell population at a volumetric ratio wherein,
i) the starting cell population is at a cell density of at least about 0.2×10⁶ cells/ml to about 5.8×10⁶cells/ml; and
ii) the volumetric ratio is 1 volume of anti-CD3/CD28 nanomatrix to 17.4 volumes or less of the starting cell population; and
b) culturing the cell population in a culture medium,
wherein the resulting T cell population comprises an increased percentage of stem memory T cells relative to a second T cell population wherein the second starting cell population of the same cell density is contacted with the anti-CD3/CD28 nanomatrix at a ratio of one volume of anti-CD3/CD28 nanomatrix to 17.5 or more volumes of the second starting cell population.

2. The method of claim 1, wherein the starting cell populations are obtained from peripheral blood, bone marrow, lymph node tissue, cord blood, thymus tissue, tissue from a site of infection, ascites, pleural effusion spleen tissue, a tumor, mesenchymal tissue, a T cell line, an induced pluriopotent stem cell, or an artificial thymic organoid (ATO) cell culture system.

3. The method of any of the preceding claims, wherein the starting cell populations are selected from the group consisting of: T lymphocytes, B lymphocytes, helper T cells, tumor infiltrating lymphocytes, memory T cells, cytotoxic T cells, natural killer T cells, peripheral blood lymphocytes, tumor infiltrating leukocytes, peripheral blood mononuclear cells, dendritic cells, cord blood stem cells, pluripotent stem cells and mesenchymal stem cells.

4. The method of any of the preceding claims, wherein the starting cell populations are peripheral blood mononuclear cells (PBMCs), positively selected CD3, CD4, or CD8 T cells or negatively selected CD3, CD4, or CD8 T cells or combinations thereof.

5. The method of any of the preceding claims, wherein the starting cell populations are peripheral blood mononuclear cells.

6. The method of any of claims 1 to 4 wherein the starting cell populations are purified T cell populations comprising CD4⁺ and/or CD8⁺ T cells.

7. The method of any of the preceding claims, wherein the starting cell density is at least about 0.50×10⁶ to about 6.00×10⁶, about 0.56×10⁶ to about 5.72×10⁶, about 0.80×10⁶ to about 4.0×10⁶, about 1.00×10⁶ to about 3.0×10⁶, about 2.00×10⁶ to about 3.5×10⁶, or about 2.50×10⁶ to about 3.5×10⁶.

8. The method of any of the preceding claims, wherein the cell density is about 2.86×10⁶ cells/ml.

9. The method of any of claims 1 to 8, wherein the starting cell density is about 1.50×10⁶ cells/ml.

10. The method of any of claims 1 to 8, wherein the starting cell density is at least about 0.28×10⁶ to about 2.86×10⁶, 0.25×10⁶ to about 2.00×10⁶, about 0.5×10⁶ to about 2.00×10⁶, about 1.00×10⁶ to about 2.00×10⁶, about 0.80×10⁶ to about 1.5×10⁶, or about 1.20×10⁶ to about 1.5×10⁶ cells/ml.

11. The method of any of claim 1 to 7 or 10, wherein the cell density is about 1.43×10⁶ cells/ml.

12. The method of any of claim 1 to 7 or 10, wherein the starting cell density is about 1.00×10⁶ cells/ml.

13. The method of any of the preceding claims wherein the volumetric ratio is about 1:5, 1:6, 1:7, 1:8, 1:9, 1:10, 1:11, 1.12, 1:13, 1:14, 1:15, 1:16 or 1:17 of anti-CD3/28 nanomatrix volume to starting cell population volume.

14. The method of any of the preceding claims wherein the starting cell populations are cultured for about 8, 9, 10, 11, 12 ,13 ,14, 15, 16, 17, 18, 19 or 20 days.

15. The method of any one of any of the preceding claims, wherein the resulting T cell population express one or more markers indicative of undifferentiated or immature T cells.

16. The method of claim 15, wherein the one or more markers indicative of undifferentiated or immature T cells are selected from the group consisting of CD62L, CD45RA, CD45RO, or any combination thereof.

17. The method of any of the previous claims wherein the resulting T cell population comprises at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% more stem memory T cells than the second resulting T cell population.

18. The method of any of the preceding claims wherein the starting cell population volumetric ratio is 1:5 and the second starting cell population volumetric ratio is 1:20, 1:25 or 1:50.

19. The method of any of claims 1 to 18, wherein the starting cell population volumetric ratio is 1:10 and the second starting cell population volumetric ratio is 1:20, 1:25 or 1:50.

20. The method of any of claims 1 to 19, wherein the starting cell population volumetric ratio is 1:15 and the second starting cell population volumetric ratio is 1:20, 1:25 or 1:50.

21. A method for increasing a percentage of stem memory T cells in a PBMC population comprising,
a) contacting a volume of an anti-CD3/CD28 nanomatrix with a volume of a starting PBMC population at a volumetric ratio wherein
i) the starting PBMC population is at a cell density of at least about 0.50×10⁶ cells/ml to about 2.00×10⁶ cells/ml; and
ii) the volumetric ratio is 1 volume of anti-CD3/CD28 nanomatrix to 5 or 10 volumes of the starting cell population; and
b) culturing the PBMC population in a culture medium for 14 to 18 days,
wherein the resulting PBMC population comprises an increased percentage of stem memory T cells relative to a second PBMC population wherein the second starting PBMC population of the same cell density is contacted with the anti-CD3/CD28 nanomatrix at a ratio of one volume of anti-CD3/CD28 nanomatrix to 20 volumes of the second starting PBMC population and cultured for the same number of days.

22. A method for increasing a percentage of stem memory T cells in a purified T cell population comprising,
a) contacting a volume of an anti-CD3/CD28 nanomatrix with a volume of a starting purified T cell population at a volumetric ratio wherein:
i) the starting T cell population is at a cell density of at least about 0.80×10⁶ cells/ml to about 1.60×10⁶ cells/ml; and
ii) the volumetric ratio is 1 volume of anti-CD3/CD28 nanomatrix to 10 or 15 volumes of the starting cell population; and
b) culturing the starting purified T cell population in a culture medium for 11 to 18 days,
wherein the resulting purified T cell population comprises an increased percentage of stem memory T cells relative to a second purified T cell population wherein the second starting purified T cell population of the same cell density is contacted with the anti-CD3/CD28 nanomatrix at a ratio of one volume of anti-CD3/CD28 nanomatrix to 20, 25 or 50 volumes of the second starting T cell population and cultured for the same number of days.

23. A method for increasing a total number of T cells in a T cell population comprising,
a) contacting a volume of an anti-CD3/CD28 nanomatrix with a volume of a starting T cell population at a volumetric ratio wherein:
i. the starting T cell population is at a cell density of at least about 0.80×10⁶ cells/ml to about 1.60×10⁶ cells/ml; and
ii. the volumetric ratio is 1 volume of anti-CD3/CD28 nanomatrix to 10 or 15 volumes of the starting cell population; and
b) culturing the T cell population in a culture medium for 11 to 18 days,
wherein the resulting T cell population comprises an increased number of T cells relative to a second T cell population wherein the second starting T cell population of the same cell density is contacted with the anti-CD3/CD28 nanomatrix at a ratio of one volume of anti-CD3/CD28 nanomatrix to 20, 25 or 50 volumes of the second starting T cell population and cultured for the same number of days.
